(19) United States
(12) Patent Application Publication
(10) Pub. No.: US 2024/0000697 A1
(43) Pub. Date: Jan. 4, 2024

(54) PERSONAL CARE COMPOSITIONS COMPRISING MICROBIALLY PRODUCED OIL

(71) Applicant: C16 Biosciences, Inc., New York, NY (US)

(72) Inventors: Harold M. MCNAMARA, Brooklyn, NY (US); Shara TICKU, New York, NY (US); David HELLER, New York, NY (US); Corentin MOEVUS, New York, NY (US); Bowen ZHANG, Monrovia, CA (US)

(21) Appl. No.: 18/035,673

(22) PCT Filed: Nov. 12, 2021
(86) PCT No.: PCT/US2021/059147

§ 371 Date: May 5, 2023

Related U.S. Application Data
(60) Provisional application No. 63/112,855, filed on Nov. 12, 2020.

(51) Int. Cl. A61K 8/92; A61K 8/37; A61Q 19/10; A61Q 19/00; C12P 7/6427; C12P 7/6409

(52) U.S. Cl. CPC A61K 8/92; A61K 8/37; A61Q 19/10; A61Q 19/00; A61Q 19/001; C12P 7/6427; C12P 7/6409; A61K 2800/85

(57) ABSTRACT

The present disclosure relates to personal care compositions comprising a microbial oil, and/or a derivative thereof. These lipids may serve as palm oil alternatives and be processed and/or derivatized by any number of means known in the art. The microbial oil or derivative thereof may be used in a variety of personal care compositions. The present disclosure also relates to methods of producing derivatives from microbial oil and producing personal care compositions comprising a microbial oil and/or a derivative thereof.

### CROSS-REFERENCE TO RELATED APPLICATIONS

This application claims the benefit of U.S. Provisional Application No. 63/112,855 filed on Nov. 12, 2020, which is hereby incorporated by reference in its entirety for all purposes.

### FIELD OF THE DISCLOSURE

The present disclosure relates to environmentally friendly and sustainable alternatives to plant-derived palm oil for personal care compositions. The palm oil alternatives are produced by oleaginous microorganisms and share one or more features with plant-derived palm oils. These alternatives may also be fractionated, treated, and/or derivatized based on their intended use.

### BACKGROUND

Palm oil is currently the most widely produced vegetable oil on the planet, as it finds uses in the manufacture of a large variety of products. It is widely used in food, and as a biofuel precursor. It's in approximately 70% of beauty care products, where the ingredient(s) is often a derivative of palm oil, such as glycerin or decyl glucoside. The global demand for palm oil is approximately 57 million tons and is steadily increasing. However, the high demand for palm oil has resulted in environmentally detrimental practices related to the expansion of plantations devoted to palm oil-producing plants. Palm oil production is a leading contributor to tropical deforestation, resulting in habitat destruction, increased carbon dioxide emissions, and local smog clouds across South East Asia.

Thus, there is an urgent need for palm oil alternatives that do not rely upon utilization of oil palms and incur the associated negative environmental costs.

### BRIEF SUMMARY

The present disclosure relates to personal care compositions comprising a microbial oil, and/or a derivative thereof, wherein the microbial oil is derived from an oleaginous yeast. The present disclosure also relates to methods for producing a personal care composition, comprising obtaining a microbial oil, and/or a derivative thereof, wherein the microbial oil or derivative is derived from an oleaginous yeast, and producing a personal care composition.

In some embodiments, the microbial oil may comprise a fatty acid profile of at least 30% saturation level, or a fatty acid profile of at least 20% palmitic acid, at least 10% stearic acid, and at least 30% oleic acid. In some embodiments, the microbial oil comprises at least one of ergosterol, β-carotene, torulene, and torularhodin. In some embodiments, the microbial oil derivative is a triglyceride, diglyceride, monoglyceride, free fatty acid, fatty acid salt, glycerin, ester, fatty alcohol, fatty amine, derivatives thereof, or combination thereof. In some embodiments, the microbial oil is refined, bleached, and/or deodorized.

In some embodiments, the present disclosure relates to microbial oil derivatives that function as a surfactant in a personal care composition. In some embodiments, the surfactant function is an emulsifier, detergent, wetting agent, foaming agent, thickening agent, emollient, pearlescent, solubilizer, conditioning agent, co-surfactant or dispersant

In some embodiments, the present disclosure relates to microbial oil derivates that function as a humectant in a personal care composition. In some embodiments, the present disclosure relates to microbial oil or derivative thereof that functions as a luxury soft oil in a personal care composition. In some embodiments, the present disclosure relates to microbial oil or derivative thereof that functions as a biologically active ingredient in a personal care composition.

In another embodiment, the present disclosure relates to personal care compositions comprising a microbial oil and/or derivative thereof, wherein the microbial oil is derived from an oleaginous yeast, and further comprising a cleaning agent, a luxury soft oil, a polymer, an essential oil, a stabilizer, an emulsifier, a thickener, an antioxidant, a biologically active ingredient, or combinations thereof. In some embodiments, the emulsifier is a polysorbate, sorbitan ester, or polyethylene glycol. In some embodiments, the cleaning agent is an alkaline solution, acidic solution, neutral solution, degreaser, scouring agent, or combinations thereof. In some embodiments, the luxury soft oil is argan oil, jojoba oil, meadowfoam seed oil, seed oil, black seed oil, evening primrose oil, walnut oil, wheat germ oil, hemp oil, rosehip oil, pumpkin seed oil, or combinations thereof. In some embodiments, the essential oil is lavender, peppermint, tea tree oil, patchouli, eucalyptus, rhododendron, or combinations thereof. In some embodiments, the biologically active ingredient is an antibiotic, antimicrobial, anti-inflammatory, antioxidant, mineral, or inorganic compound derived from a mineral. In some embodiments, the biologically active ingredient is zinc oxide, retinol, or salicylic acid. In some embodiments, the composition does not comprise palm oil or palm kernel oil or derivatives thereof. In some embodiments, the composition is a solid, liquid, cream, lotion, spray, gel, or foam. In some embodiments, the personal care item is a soap, body lotion, face lotion, luxury soft oil, cleansing oil, cream, deodorant, or hair care item. In some embodiments, the oleaginous yeast is Rhodosporidium toruloides.

In another embodiment, the present disclosure relates to a personal care composition comprising an oil and/or a derivative thereof, wherein said oil and/or derivative thereof consists of a microbial oil and/or derivative produced by an oleaginous yeast. In some embodiments, the composition comprises triglycerides, wherein greater than 40% of the triglycerides have one unsaturated sidechain, and wherein greater than 30% of the triglycerides have two unsaturated sidechains.

In another embodiment, the present disclosure relates to a personal care composition comprising sodium stearate derived from a stearic acid produced by an oleaginous yeast. In another embodiment, the present disclosure relates to a personal care composition comprising a fatty acid-ingredient derived from an oleaginous yeast. In another embodiment, the present disclosure relates to a personal care composition comprising an isostearyl palmitate derived from an oleaginous yeast. In another embodiment, the present disclosure relates to a personal care composition comprising cetearyl alcohol derived from fatty alcohols produced by an oleaginous yeast.

### BRIEF DESCRIPTION OF THE DRAWINGS

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIG. 1 is a flow diagram illustrating examples of various methods of processing the microbial oil and the resulting derivatives (oleochemicals) which may be used in personal care compositions.

FIG. 2A shows a chromatogram of the triglyceride composition analysis of exemplary crude microbial oil; FIG. 2B shows a chromatogram of the triglyceride composition analysis of exemplary crude palm oil; and FIG. 2C shows a chromatogram of the triglyceride composition analysis of exemplary crude hybrid palm oil.

FIG. 3 is a flow diagram of fractions produced from microbial oil.

FIG. 4A is a photograph of a fractionation of crude microbial oil (left) and crude palm oil (right). FIG. 4B is a photograph of a complete fractionation of crude microbial oil. FIG. 4C is a photograph of an incomplete fractionation of crude microbial oil.

FIG. 5 is a bar graph showing gas chromatography-mass spectrometry (GCMS) data highlighting how the fractionation shifts the fatty acid profile in the olein and stearin layers.

FIG. 6 is a bar graph of the data shown in FIG. 5 illustrating the overall balance of saturated fatty acids (SFA), monounsaturated fatty acids (MUFA), and polyunsaturated fatty acids (PUFA).

FIG. 7A shows a chromatogram of the fatty acid composition analysis of exemplary crude microbial oil; FIG. 7B shows a chromatogram of the fatty acid composition analysis of exemplary crude palm oil; FIG. 7C shows a chromatogram of the fatty acid composition analysis of exemplary crude hybrid palm oil; and FIG. 7D shows a bar graph of representative fatty acid compositions of microbial oil and palm oil.

FIG. 8 is a flow diagram of fatty acids produced from microbial oil.

FIG. 9 is a flow diagram of fatty alcohols produced from microbial oil.

FIG. 10 shows a chromatogram of the tocopherols analysis of exemplary crude microbial oil, crude palm oil, and crude hybrid palm oil. Notable peaks are annotated, with “External ISTD” illustrating the location of the standard.

FIG. 11A shows a photograph of cleansing oils made with microbial oil. The bottle shown on the left also comprises a derivative of the microbial oil, whereas the bottle on the right comprises the same derivative from palm.

FIGS. 11B-11D are photographs showing how the cleaning oils can remove makeup.

FIG. 12 shows a photograph of repair serums made with microbial oil. The bottle shown on the left also comprises a derivative of the microbial oil, whereas the bottle on the right comprises the same derivative from palm.

FIGS. 13A-13C show photographs of lip balms made with microbial oil. In each image, the bottle or tube shown on the left also comprises a derivative of the microbial oil, whereas the bottle or tube on the right comprises the same derivative from palm.

### DETAILED DESCRIPTION

The following description includes information that may be useful in understanding the present disclosure. It is not an admission that any of the information provided herein is prior art or relevant to the presently claimed disclosures, or that any publication specifically or implicitly referenced is prior art.

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

As used herein, the singular forms “a,” “an,” and “the: include plural referents unless the content clearly dictates otherwise.

The term “about” or “approximately” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

“Cleaning agent” as used herein is any substance used to remove dirt, dust, stains, and/or odor. They may also be classified as a disinfectant or anti-microbial.

A “fatty acid” is a carboxylic acid with a long aliphatic chain, which is either saturated or unsaturated. Most naturally occurring fatty acids have an unbranched chain of an even number of carbon atoms, from 4 to 28. Fatty acids are usually not found free in organisms, but instead within three main classes of esters: triglycerides, phospholipids, and cholesteryl esters. Within the context of this disclosure, a reference to a fatty acid may refer to either its free or ester form.

“Fatty acid profile” as used herein refers to how specific fatty acids contribute to the chemical composition of an oil.

“Fatty acid-ingredient” as used herein refers to a cosmetic grade ingredient. For example, whereas pure “stearic acid” is C18:0, in cosmetics when “stearic acid” is listed as an ingredient, it is a mixture of C16:0/C18:0.

As used herein, the term “fractionable” is used to refer to a microbial oil or lipid composition which can be separated into at least two fractions that differ in saturation levels and wherein the at least two fractions each make up at least 10% w/w (or mass/mass) of the original microbial oil or lipid composition. The saturation levels of the fractions may be characterized by, e.g., their iodine value (IV). The IV of the fractions may differ by at least 10. Accordingly, a “fraction” as used herein refers to a separable component of a microbial oil that differs in saturation level from at least one other separable component of the microbial oil.

“Lipid” means any of a class of molecules that are soluble in nonpolar solvents (such as ether and hexane) and relatively or completely insoluble in water. Lipid molecules have these properties, because they are largely composed of long hydrocarbon tails that are hydrophobic in nature. Examples of lipids include fatty acids (saturated and unsaturated); glycerides or glycerolipids (such as monoglycerides, diglycerides, triglycerides or neutral fats, and phosphoglycerides or glycerophospholipids); and nonglycerides (sphingolipids, tocopherols, tocotrienols, sterol lipids including cholesterol and steroid hormones, prenol lipids including terpenoids, fatty alcohols, waxes, and polyketides).

“Luxury soft oil” or “soft oil” refers to oils that are liquid at room temperature.

“Microorganism” and “microbe” mean any microscopic unicellular organism and can include bacteria, algae, yeast, or fungi.

“Oleaginous” as used herein refers to material, e.g., a microorganism, which contains a significant component of oils, or which is itself substantial composed of oil. An oleaginous microorganism can be one that is naturally occurring or synthetically engineered to generate a significant proportion of oil.

“Oleaginous yeast” as used herein refers to a collection of yeast species that can accumulate a high proportion of their biomass as lipids (namely greater than 20% of dry cell mass). An oleaginous yeast can be one that is naturally occurring or synthetically engineered to generate a significant proportion of oil.

“Personal care composition”, “personal care item” or “personal care product” as used herein relate to a broad category of compositions including, for example, cosmetics, cosmeceuticals, skin care products and treatments, hair care products, cleansers, antiperspirants and deodorants, toothpastes and oral rinses, nail treatments and nail polishes, and perfumes.

“Polymer” as used herein refers to a broad category of substances composed of the same, or similar, repeating subunits (monomers), and may be natural or synthetic.

As used herein, “RBD” refers to refinement, bleaching, and deodorizing or refers to an oil that has undergone these processes.

“Rhodosporidium toruloides” refers to a particular species of oleaginous yeast. Previously called Rhodotorula glutinis or Rhodotorula gracilis. Also abbreviated as R. toruloides. This species includes multiple strains with minor genetic variation.

For the purposes of this disclosure, “single cell oils,” “microbial oils,” “lipid composition” and “oils” refer to microbial lipids produced by oleaginous microorganisms.

“Surfactants” as used herein refers to a broad category of compounds that lower the surface tension between two liquids, for example oil and water, between a gas and a liquid, or between a liquid and a solid. In some instances, they can act as an anti-microbial agent and/or a preservative.

“Tailored fatty acid profile” as used herein refers to a fatty acid profile in a microbial oil which has been manipulated towards target properties, either by changing culture conditions, the species of oleaginous microorganism producing the microbial oil, or by genetically modifying the oleaginous microorganism.

“Triglyceride(s)” as used herein refers to a glycerol bound to three fatty acid molecules. They may be saturated or unsaturated, and various denominations may include other isomers. For example, reference to palmitic-oleic-palmitic (P-O-P) would also include the isomers P-P-O and O-P-P.

“W/W” or “w/w”, in reference to proportions by weight, refers to the ratio of the weight of one substance in a composition to the weight of the composition. For example, reference to a composition that comprises 5% w/w oleaginous yeast biomass means that 5% of the composition's weight is composed of oleaginous yeast biomass (e.g., such a composition having a weight of 100 mg would contain 5 mg of oleaginous yeast biomass) and the remainder of the weight of the composition (e.g., 95 mg in the example) is composed of other ingredients.

### Overview

The present disclosure relates to personal care compositions comprising a microbial oil or derivative thereof. These lipids may serve as palm oil alternatives and be processed and/or derivatized by any number of means known in the art. For example, the microbial oil or derivative thereof may be a triglyceride, diglyceride, monoglyceride, free fatty acid, fatty acid salt, glycerol, ester, fatty alcohol, fatty amine, derivatives thereof, or combinations thereof. The microbial oil or derivative thereof may be used in a variety of personal care products, including, for example, cleansers, hair care products, lotions, deodorants, soaps, toothpaste, oral rinses, nail polishes, nail treatments, cosmetics, and cosmeceuticals. The present disclosure also relates to methods of producing compositions comprising a microbial oil or derivative thereof. In some embodiments, the microbial oil is derived from an oleaginous yeast.

### Oleaginous Microorganisms

An embodiment of the present disclosure relates to personal care compositions comprising a microbial oil, or derivative thereof, derived from an oleaginous microorganism.

The use of oleaginous microorganisms for lipid production has many advantages over traditional oil harvesting methods, e.g., palm oil harvesting from palm plants. For example, microbial fermentation (1) does not compete with food production in terms of land utilization; (2) can be carried out in conventional microbial bioreactors; (3) has rapid growth rates; (4) is unaffected or minimally affected by space, light, or climate variations; (5) can utilize waste products as feedstock; (6) is readily scalable; and (7) is amenable to bioengineering for the enrichment of desired fatty acids or oil compositions. In some embodiments, the present methods have one or more of the aforementioned advantages over plant-based oil harvesting methods.

In some embodiments, the oleaginous microorganism is an oleaginous microalgae. In some embodiments, the microalgae is of the genus Botryococcus, Cylindrotheca, Nitzschia, or Schizochytrium. In some embodiments, the oleaginous microorganism is an oleaginous bacterium. In some embodiments, the bacterium is of the genus Arthrobacter, Acinetobacter, Rhodococcus, or Bacillus. In some embodiments, the bacterium is of the species Acinetobacter calcoaceticus, Rhodococcus opacus, or Bacillus alcalophilus. In some embodiments, the oleaginous microorganism is an oleaginous fungus. In some embodiments, the fungus is of the genus Aspergillus, Mortierella, or Humicola. In some embodiments, the fungus is of the species Aspergillus oryzae, Mortierella isabellina, Humicola lanuginosa, or Mortierella vinacea.

Oleaginous yeast in particular are robust, viable over multiple generations, and versatile in nutrient utilization. They also have the potential to accumulate intracellular lipid content up to greater than 70% of their dry biomass. In some embodiments, the oleaginous microorganism is an oleaginous yeast. In some embodiments, the yeast may be in haploid or diploid forms. The yeasts may be capable of undergoing fermentation under anaerobic conditions, aerobic conditions, or both anaerobic and aerobic conditions. A variety of species of oleaginous yeast that produce suitable oils and/or lipids can be used to produce microbial lipids in accordance with the present disclosure. In some embodiments, the oleaginous yeast naturally produces high (20%, 25%, 50% or 75% of dry cell weight or higher) levels of suitable oils and/or lipids. Considerations affecting the selection of yeast for use in the invention include, in addition to production of suitable oils or lipids for production of food products: (1) high lipid content as a percentage of cell weight; (2) ease of growth; (3) ease of propagation; (4) ease of biomass processing; and (5) glycerolipid profile. In some embodiments, the oleaginous yeast comprise cells that are capable of producing at least 20%, 25%, 50% or 75% or more lipid by dry weight. In other embodiments, the oleaginous yeast contains at least 25-35% or more lipid by dry weight.

Suitable species of oleaginous yeast for producing the microbial lipids of the present disclosure include, but are not limited to Candida apicola, Candida sp., Cryptococcus albidus. Cryptococcus curvatus, Cryptococcus terricolus, Cutaneotrichosporon oleaginosus, Debaromyces hansenii, Endomycopsis vernalis, Geotrichum carabidarum, Geotrichum cucujoidarum, Geotrichum histeridarum, Geotrichum silvicola, Geotrichum vulgare, Hyphopichia burtonii, Lipomyces hpofer, Lypomyces orentalis, Lipomyces starkeyi, Lipomyces tetrasporous, Pichia mexicana, Rodosporidium sphaerocarpum, Rhodosporidium toruloides Rhodotorula aurantiaca, Rhodotorula dairenensis, Rhodotorula diffluens, Rhodotorula glutinus, Rhodotorula glutinis var. glutinis, Rhodotorula gracilis, Rhodotorula graminis Rhodotorula minuta, Rhodotorula mucilaginosa, Rhodotorula mucilaginosa, Rhodotorula terpenoidahs, Rhodotorula toruloides, Sporobolomyces alborubescens, Starmerella bombicola, Torulaspora delbruekii, Torulaspora pretoriensis, Trichosporon behrend, Trichosporon brassicae, Trichosporon domesticum, Trichosporon laibachii, Trichosporon loubieri, Trichosporon loubieri, Trichosporon montevideense, Trichosporon pullulans, Trichosporon sp., Wickerhamomyces canadensis, Yarrowia hpolytica, and Zygoascus meyerae.

In some embodiments, the yeast is of the genera Yarrowia, Candida, Rhodotorula, Rhodosporidium, Metschnikowia, Cryptococcus, Trichosporon, or Lipomyces. In some embodiments, the yeast is of the genus Yarrowia. In some embodiments, the yeast is of the species Yarrowia hpolytica. In some embodiments, the yeast is of the genus Candida. In some embodiments, the yeast is of the species Candida curvata. In some embodiments, the yeast is of the genus Cryptococcus. In some embodiments, the yeast is of the species Cryptococcus albidus. In some embodiments, the yeast is of the genus Lipomyces. In some embodiments, the yeast is of the species Lipomyces starkeyi. In some embodiments, the yeast is of the genus Rhodotorula. In some embodiments, the yeast is of the species Rhodotorula glutinis. In some embodiments, the yeast is of the genus Metschnikowia. In some embodiments, the yeast is of the species Metschnikowia pulcherrima.

In some embodiments, the oleaginous yeast is of the genus Rhodosporidium. In some embodiments, the yeast is of the species Rhodosporidium toruloides. In some embodiments, the oleaginous yeast is of the genus Lipomyces. In some embodiments, the oleaginous yeast is of the species Lipomyces Starkeyi.

In some embodiments, the oleaginous microorganisms that produce the microbial lipids of the present disclosure are a homogeneous population comprising microorganisms of the same species and strain. In some embodiments, the oleaginous microorganisms that produce the microbial lipids of the present disclosure are a heterogeneous population comprising microorganisms from more than one strain. In some embodiments, the oleaginous microorganisms that produce the microbial lipids of the present disclosure are a heterogeneous population comprising two or more distinct populations of microorganisms of different species.

The oleaginous microorganisms that produce the microbial lipids used in the compositions of matter of the present disclosure may have been improved in terms of one or more aspects of lipid production. These aspects may include lipid yield, lipid titer, dry cell weight titer, lipid content, and lipid composition. In some embodiments, lipid production may have been improved by genetic or metabolic engineering to adapt the microorganism for optimal growth on the feedstock. In some embodiments, lipid production may have been improved by varying one or more parameters of the growing conditions, such as temperature, shaking speed, growth time, etc. The oleaginous microorganisms of the present disclosure, in some embodiments, are grown from isolates obtained from nature (e.g., wild-types). In some embodiments, wild-type strains are subjected to natural selection to enhance desired traits (e.g., tolerance of certain environmental conditions such as temperature, inhibitor concentration, pH, oxygen concentration, nitrogen concentration, etc.). For example, a wild-type strain (e.g., yeast) may be selected for its ability to grow and/or ferment in a feedstock of the present disclosure, e.g., a feedstock comprising one or more microorganism inhibitors. In other embodiments, wild-type strains are subjected to directed evolution to enhance desired traits (e.g., lipid production, inhibitor tolerance, growth rate, etc.). In some embodiments, the cultures of microorganisms are obtained from culture collections exhibiting desired traits. In some embodiments, strains selected from culture collections are further subjected to directed evolution and/or natural selection in the laboratory. In some embodiments, oleaginous microorganisms are subjected to directed evolution and selection for a specific property (e.g., lipid production and/or inhibitor tolerance). In some embodiments, the oleaginous microorganism is selected for its ability to thrive on a feedstock of the present disclosure.

In some embodiments, directed evolution of the oleaginous microorganisms generally involves three steps. The first step is diversification, wherein the population of organisms is diversified by increasing the rate of random mutation creating a large library of gene variants. Mutagenesis can be accomplished by methods known in the art (e.g., chemical, ultraviolet light, etc.). The second step is selection, wherein the library is tested for the presence of mutants (variants) possessing the desired property using a screening method. Screens enable identification and isolation of high-performing mutants. The third step is amplification, wherein the variants identified in the screen are replicated. These three steps constitute a “round” of directed evolution. In some embodiments, the microorganisms of the present disclosure are subjected to a single round of directed evolution. In other embodiments, the microorganisms of the present disclosure are subjected to multiple rounds of directed evolution. In various embodiments, the microorganisms of the present disclosure are subjected to 2, 3, 4, 5, 6, 7, 8, 9, 10, 20, 30, 40, 50, 60, 70, 80, 90, or 100 or more rounds of directed evolution. In each round, the organisms expressing the highest level of the desired trait of the previous round are diversified in the next round to create a new library. This process may be repeated until the desired trait is expressed at the desired level.

### Properties of Microbial Oil

The present disclosure provides microbial oils and derivatives thereof. These lipids may serve as palm oil alternatives and may be processed and/or derivatized by any number of means known in the art. The microbial oil and/or derivatives thereof may be used in a variety of downstream products of interest, such as personal care compositions.

An embodiment of the present disclosure relates to personal care compositions comprising a microbial oil, and/or a derivative thereof, wherein the microbial oil is derived from an oleaginous yeast. In some embodiments, the derivative is a triglyceride, diglyceride, monoglyceride, free fatty acid, fatty acid salt, glycerin, ester, fatty alcohol, fatty amine, derivatives thereof, or combination thereof. In some embodiments, the microbial oil comprises a fatty acid profile of at least 20% palmitic acid, at least 10% stearic acid, and at least 30% oleic acid. In some embodiments, the microbial oil comprises at least one of ergosterol, β-carotene, torulene, and torularhodin.

### Sterol Composition

In some embodiments, the microbial oil comprises one or more sterols. In some embodiments, the microbial oil comprises ergosterol. In some embodiments, the microbial oil comprises at least 50 ppm ergosterol. In some embodiments, the microbial oil comprises at least 100 ppm ergosterol.

In some embodiments, the microbial oil comprises less than 100 ppm of a phytosterol, cholesterol, or a protothecasterol. In some embodiments, the microbial oil comprises less than 50 ppm of of a phytosterol, cholesterol, or a protothecasterol. In some embodiments, the microbial oil does not comprise a sterol selected from a phytosterol, cholesterol, or a protothecasterol.

In some embodiments, the microbial oil does not comprise plant sterols. In some embodiments, the microbial oil does not comprise one or more phytosterols. In some embodiments, the microbial oil does not comprise campesterol, β-sitosterol, or stigmasterol. In some embodiments, the microbial oil does not comprise cholesterol. In some embodiments, the microbial oil does not comprise protothecasterol.

### Pigments

In some embodiments, the microbial oil comprises a pigment. In some embodiments, the microbial oil comprises at least one pigment selected from the group consisting of carotene, torulene and torulorhodin. In some embodiments, the microbial oil comprises carotene. In some embodiments, the microbial oil comprises torulene. In some embodiments, the microbial oil comprises torulorhodin. In some embodiments, the microbial oil comprises each of carotene, torulene and torulorhodin. In some embodiments, the microbial oil does not comprise chlorophyll.

### Fatty Acid Composition

The composition of the microbial oil may vary depending on the strain of microorganism, feedstock composition, and growing conditions. In some embodiments, the microbial oil produced by the oleaginous microorganisms of the present disclosure comprise about 90% w/w triacylglycerol with a percentage of saturated fatty acids (% SFA) of about 44%. The most common fatty acids produced by oleaginous microbial fermentation on the present feedstocks are oleic acid (C18:1), stearic acid (C18:0), palmitic acid (C16:0), palmitoleic acid (C16:1), and myristic acid (C14:0).

In some embodiments, the microbial oil comprises myristic acid (C14:0). In some embodiments, the microbial oil comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% myristic acid.

In some embodiments, the microbial oil comprises at least 5%, at least 10%, at least 15%, at least 20%, at least 25%, at least 30%, at least 35%, at least 40%, at least 45%, at least 50%, at least 55%, or at least 60% w/w palmitic acid (C16:0). In some embodiments, the microbial oil comprises at least 5% w/w palmitic acid. In some embodiments, the microbial oil comprises at least 10% w/w palmitic acid. In some embodiments the microbial oil comprises 10-20% w/w palmitic acid. In some embodiments the microbial oil comprises 13-16% w/w palmitic acid.

In some embodiments, the microbial oil comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9% or at least 10% w/w palmitoleic acid (C16:1). In some embodiments, the microbial oil comprises at least 0.1% w/w palmitoleic acid. In some embodiments, the microbial oil comprises at least 0.5% w/w palmitoleic acid. In some embodiments, the microbial oil comprises 0.5-10% w/w palmitoleic acid. In some embodiments, the microbial oil comprises 1-5% w/w palmitoleic acid.

In some embodiments, the microbial oil comprises margaric acid (C17:0). In some embodiments, the microbial oil comprises at least 1%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or at least 25% margaric acid. In some embodiments, the microbial oil comprises 5-25% w/w margaric acid. In some embodiments, the microbial oil comprises 9-21% w/w margaric acid.

In some embodiments, the microbial oil comprises at least 1%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or at least 25% w/w stearic acid (C18:0). In some embodiments, the microbial oil comprises at least 1% w/w stearic acid. In some embodiments, the microbial oil comprises at least 5% w/w stearic acid. In some embodiments, the microbial oil comprises 5-25% w/w stearic acid. In some embodiments, the microbial oil comprises 9-21% w/w stearic acid.

In some embodiments, the microbial oil comprises at least 5%, at least 10%, at least 15%, at least 20%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54% at least 55%, at least 56%, at least 57%, at least 58%, at least 59%, or at least 60% w/w oleic acid (C18:1). In some embodiments, the microbial oil comprises at least 25% w/w oleic acid. In some embodiments, the microbial oil comprises at least 30% w/w oleic acid. In some embodiments, the microbial oil comprises 30-65% w/w oleic acid. In some embodiments, the microbial oil comprises 39-55% w/w oleic acid.

In some embodiments, the microbial oil comprises C18:2 (linoleic acid). In some embodiments, the microbial oil comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% linoleic acid.

In some embodiments, the microbial oil comprises C18:3 (linolenic acid). In some embodiments, the microbial oil comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% linolenic acid.

In some embodiments, the microbial oil comprises C20:0 (arachidic acid). In some embodiments, the microbial oil comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% arachidic acid.

In some embodiments, the microbial oil comprises C24:0 (lignoceric acid). In some embodiments, the microbial oil comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% lignoceric acid.

In some embodiments, the microbial oil comprises C12:0. In some embodiments, the microbial oil comprises C15:1. In some embodiments, the microbial oil comprises C16:1. In some embodiments, the microbial oil comprises C17:1. In some embodiments, the microbial oil comprises C18:3. In some embodiments, the microbial oil comprises C20:1. In some embodiments, the microbial oil comprises C22:0. In some embodiments, the microbial oil comprises C22:1. In some embodiments, the microbial oil comprises C22:2. In some embodiments, the microbial oil comprises about 0.1%, about 0.2%, about 0.3%, about 0.4%, about 0.5%, about 0.6%, about 0.7%, about 0.8%, about 0.9%, about 1%, about 2%, about 3%, about 4%, or about 5% of any one of these fatty acids. In some embodiments, the microbial oil comprises about 0-5% of any one of these fatty acids. In some embodiments, the microbial oil comprises about 0.1-2% of any one of these fatty acids.

### Characteristics Similar to Plant-Derived Palm Oil

In some embodiments, the microbial oils of the present disclosure have differences from plant-derived palm oil. In some embodiments, these differences are useful and allow for manipulation of the microbial oil for the improved production of a given product compared to plant-derived palm oil. For example, in some embodiments, the fatty acid profile of a microbial oil is tailored so as to produce a higher fraction of one or more fatty acids of interest for use in production of a product. In some embodiments, other parameters of the microbial oil are also able to be manipulated for increased production of a component of interest or decreased production of an undesired component relative to plant-derived palm oil.

However, in some embodiments, the present compositions are also useful as environmentally friendly alternatives to plant-derived palm oil. Therefore, in some embodiments, the microbial oil has one or more properties similar to those of plant-derived palm oil. Exemplary properties include apparent density, refractive index, saponification value, unsaponifiable matter, iodine value, slip melting point, and fatty acid composition.

In some embodiments, the microbial oil has a fatty acid profile similar to that of plant-derived palm oil. In some embodiments, the microbial oil has a significant fraction of C16:0 fatty acid. In some embodiments, the microbial oil has a significant fraction of C18:1 fatty acid. In some embodiments, the microbial oil comprises 10-45% C16 saturated fatty acid. In some embodiments, the microbial oil comprises 10-70% C18 unsaturated fatty acid.

In some embodiments, the microbial oil has a similar ratio of saturated to unsaturated fatty acids as plant-derived palm oil. Some plant-derived palm oils have approximately 50% of each. In some embodiments, the microbial oil has a saturated fatty acid composition of about 50% and an unsaturated fatty acid composition of about 50%. In some embodiments, the microbial oil has a saturated fatty acid composition of about 40-60% and an unsaturated fatty acid composition of about 40-60%. In some embodiments, the microbial oil has a saturated fatty acid composition of about 30-70% and an unsaturated fatty acid composition of about 30-70%. In some embodiments, the microbial oil has about 30%, 35%, 40%, 45%, 50%, 55%, 60%, 65%, or 70% saturated fatty acids.

In some embodiments, the microbial oil has a similar level of mono-unsaturated fatty acids as plant-derived palm oil. Some plant-derived palm oils contain approximately 40% mono-unsaturated fatty acids. In some embodiments, the microbial oil contains about 40% mono-unsaturated fatty acids. In some embodiments, the microbial oil contains about 30-50% mono-unsaturated fatty acids. In some embodiments, the microbial oil contains about 5-60% mono-unsaturated fatty acids. In some embodiments, the microbial oil has about 5%, 10%, 15%, 20%, 25%, 30%, 35%, 40%, 45%, 50%, 55%, or 60% mono-unsaturated fatty acids.

In some embodiments, the microbial oil has a similar level of poly-unsaturated fatty acids as plant-derived palm oil. Some plant-derived palm oils contain approximately 10% poly-unsaturated fatty acids. In some embodiments, the microbial oil contains about 10% poly-unsaturated fatty acids. In some embodiments, the microbial oil contains about 5-25% poly-unsaturated fatty acids. In some embodiments, the microbial oil has about 5%, 6%, 7%, 8%, 9%, 10%, 11%, 12%, 13%, 14%, 15%, 16%, 17%, 18%, 19%, 20%, 21%, 22%, 23%, 24%, or 25% poly-unsaturated fatty acids.

In some embodiments, the microbial oil has a similar iodine value as plant-derived palm oil. Some plant-derived palm oils have an iodine value of about 50.4-53.7. In some embodiments, the microbial oil has an iodine value of about 49-65. In some embodiments, the microbial oil has an iodine value of about 49, 50, 51, 52, 53, 54, 55, 56, 57, 58, 59, 60, 61, 62, 63, 64, or 65.

Table 1 shows ranges for the fatty acid composition of an illustrative plant-derived palm oil and ranges of values for the fatty acid composition of illustrative microbial oil. In some embodiments, the microbial oil has one or more fatty acid composition parameters similar to those of Table 1. For example, in some embodiments, the microbial oil has a value within the plant-derived palm oil range for a given fatty acid composition parameter. In some embodiments, the microbial oil has a value within the microbial oil ranges provided in Table 1 for one or more parameters.

TABLE 1
Illustrative fatty acid compositions of microbial oil

| | Illustrative | Illustrative |
|---|---|---|
| | plant-derived | microbial oil |
| Component | palm oil range | range |
| C8:0 | 0.0-0.1% | 0.0% |
| C10:0 | 0.0-0.1% | 0.0-0.1% |
| C12:0 | 0.0-0.5% | 0.0-0.5% |
| C14:0 | 0.5-2.0% | 0.0-5.0% |
| C14:1c | 0.0-0.1% | 0.0-0.2% |
| C15:1 | 0.0-0.1% | 0.0-1.0% |
| C16:0 | 39.3-47.5% | 10.0-50.0% |
| C16:1 | 0.0-0.6% | 0.0-1.0% |
| C17:0 | 0.0-0.2% | 0.0-15.0% |
| C17:1 | 0.0-0.1% | 0.0-0.1% |
| C18:0 | 3.5-6.0% | 7.0-35.0% |
| C18:1 | 36.0-44.0% | 10.0-50.0% |
| C18:2 | 9.0-12.0% | 8.0-20.0% |
| C18:3 | 0.0-0.5% | 0.0-0.5% |
| C20:0 | 0.0% | 0.0-10.0% |
| C20:1 | 0.0-0.4% | 0.0-5.0% |
| C22:0 | 0.0-0.2% | 0.0-5.0% |
| C22:1 | 0.0% | 0.0-1.0% |
| C22:2 | 0.0% | 0.0-5.0% |
| C24:0 | 0.0% | 0.0-10.0% |

In some embodiments, the microbial oil has a similar slip melting point to plant-derived palm oil. Some plant-derived palm oils have a slip melting point of about 33.8-39.2° C. In some embodiments, the microbial oil has a slip melting point of about 30-40° C. In some embodiments, the microbial oil has a slip melting point of about 30, 31, 32, 33, 34, 35, 36, 37, 38, 39, or 40° C.

In some embodiments, the microbial oil has a saponification value similar to that of plant-derived palm oil. Some plant-derived palm oils have a saponification value of about 190-209. In some embodiments, the microbial oil has a saponification value of about 150-210. In some embodiments, the microbial oil has a saponification value of about 150, 155, 160, 165, 170, 175, 180, 185, 190, 195, 200, 205, or 210.

In some embodiments, the microbial oil has a similar unsaponifiable matter content to that of plant-derived palm oil. Some plant-derived palm oils have an unsaponifiable matter content of about 0.19-0.44% by weight. In some embodiments, the microbial oil has an unsaponifiable matter content of less than 5% by weight.

In some embodiments, the microbial oil has a similar refractive index to that of plant-derived palm oil. Some plant-derived palm oils have a refractive index of about 1.4521-1.4541. In some embodiments, the microbial oil has a refractive index of about 1.3-1.6.

In some embodiments, the microbial oil has a similar apparent density to that of plant-derived palm oil. Some plant-derived palm oils have an apparent density of about 0.8889-0.8896. In some embodiments, the microbial oil has an apparent density of about 0.88-0.9.

In some embodiments, the microbial oil has one or more parameters similar to those of hybrid palm oil.

In some embodiments, the microbial oil may be used as a palm oil substitute or alternative. In some embodiments, the microbial oil may be used in the manufacture of any product for which palm oil can be employed. For example, in some embodiments, the microbial oil may be used in a personal care composition. In some embodiments, the microbial oil comprises a fatty acid profile of at least 30% saturation level. In some embodiments, the microbial oil comprises a fatty acid profile of at least 20% palmitic acid, at least 10% stearic acid, and at least 30% oleic acid. In some embodiments, the composition further comprises at least one of ergosterol, β-carotene, torulene, and torularhodin. In some embodiments, the composition does not comprise palm oil or palm kernel oil or derivatives thereof. In some embodiments, the disclosure teaches methods of producing a personal care composition, comprising providing a microbial oil, or derivative thereof, and producing a personal care composition. In some embodiments, the personal care composition is a solid, liquid, cream, lotion, spray, gel, or foam. In some embodiments, the personal care composition is a soap, body lotion, face lotion, luxury soft oil, cleansing oil, cream, deodorant, or hair care item.

### Triglyceride Composition

Tables 2A and 2B show ranges for the triglyceride composition of an illustrative plant-derived palm oil and ranges of values for the triglyceride composition of illustrative microbial oil. The abbreviations used are as follows: S: Stearic fatty acid; P: Palmitic fatty acid; O: Oleic fatty acid. For each component shown below in Table 2A, for example P-O-P, the corresponding measurements for that molecule may also include other isomers, for example P-P-0 and 0-P-P. In some embodiments, the microbial oil has one or more triglyceride composition parameters similar to those of Table 2A and Table 2B. For example, in some embodiments, the microbial oil has a value similar to or within the plant-derived palm oil range for a given triglyceride composition parameter. For example, plant-derived palm oil has an O-O-P of approximately 23.24% and microbial-derived oil has an 0-0-P of approximately 20.78.

In some embodiments, the microbial oil has a similar triglyceride content to that of plant-derived palm oil. For example, the total triglyceride content of sat-unsat-sat in plant-derived palm oil is approximately 49.53 and microbial-derived oil has approximately 49.42. In some embodiments, the microbial oil has a value different than plant-derived palm oil. For example, plant-derived palm oil has approximately 9.04% sat-sat-sat chains, whereas microbial-derived oil has approximately 3.36%. Some plant-derived palm oils have a triglyceride content of over 95%. In some embodiments, the microbial oil has a triglyceride content of 90-98%. In some embodiments, the microbial oil has a triglyceride content of about 90, 91, 92, 93, 94, 95, 96, 97, or 98%.

TABLE 2A
Illustrative triglyceride compositions of microbial oil

| | Crude plant- | Crude |
|---|---|---|
| | derived palm oil | microbial oil |
| Component | range | range |
| P-P-P | 6.48 +/− 1.62 | 1.02 +/− 0.25 |
| P-P-O | 31.62 +/− 7.9 | 22.53 +/− 5.63 |
| O-O-P | 23.24 +/− 5.81 | 20.78 +/− 5.12 |
| S-O-S | 0.6 +/− 0.15 | 1.53 +/− 0.38 |
| S-O-O | 2.46 +/− 0.62 | 4.29 +/− 1.07 |
| P-O-S | 6.11 +/− 1.53 | 10.25 +/− 2.56 |
| M-O-P | 1.58 +/− 0.40 | 4.73 +/− 1.18 |
| Sat-Sat-Sat | 9.04 +/− 1.36 | 3.36 +/− 0.50 |
| Sat-Unsat-Sat | 49.53 +/− 7.43 | 49.42 +/− 7.41 |
| Sat-Unsat-Unsat | 36.66 +/− 5.50 | 39.42 +/− 5.91 |
| Unsat-Unsat-Unsat | 4.77 +/− 0.72 | 6.86 +/− 1.03 |

TABLE 2B
Summary total triglyceride compositions

| | Number of unsaturated side chains | | | | |
|---|---|---|---|---|---|
| | 0 | 1 | 2 | 3 | total |
| Crude Plant-derived | 9.04% | 49.53% | 36.66% | 4.76887% | 100.00 |
| palm oil | | | | | |
| Crude Microbial- | 3.36% | 49.42% | 39.42% | 6.86% | 99.06 |
| derived oil | | | | | |

In some embodiments, the microbial oil has a similar diacylglycerol content as a plant-derived palm oil. Percentage of diacylglycerol varies between about 4-11% for some plant-derived palm oils. In some embodiments, the microbial oil comprises 0-15% diacylglycerol content.

In some embodiments, the microbial oil has a similar triacylglycerol profile to plant-derived palm oil. Some plant-derived palm oils have over 80% C50 and C52 triacylgylcerols. In some embodiments, the microbial oil has a triacylglycerol profile comprising at least 40% C50 and C52 triacylglycerols.

In some embodiments, the microbial oil has a triglyceride profile wherein greater than 40% of the triglycerides have one unsaturated sidechain, and wherein greater than 30% of the triglycerides have two unsaturated sidechains

### Oxidative Stability

The unsaturated lipids in vegetable oils are susceptible to oxidation over time, which can be accelerated when the oil is exposed to heat, light, or metals. Oxidation causes changes in the chemical, sensory, and nutritional properties of the oil, and can result in, among other things, an unpleasant odor.

The oxidative stability of the microbial oil described herein was analyzed by detection of peroxide using methods known in the art, for example, by titration reaction of iodine and peroxide with a starch indicator. The peroxide value of the microbial oil was less than 2 mEq/kg, which is within the Malaysian Palm Oil Board (MPOB) specification.

### Processing of Microbial Oil

In some embodiments, once the microbial oil is obtained from the oleaginous microorganism, it is subjected to some form of processing. In some embodiments, the microbial oil is refined, bleached, deodorized, fractionated, treated, and/or derivatized.

In some embodiments, the microbial oil is refined. In some embodiments, prior to refinement, the microbial oil is referred to as crude microbial oil. In some embodiments, the refinement process comprises the removal of one or more non-triacylglycerol components. Typical non-triacylglycerol components removed or reduced via oil refinement include free fatty acids, partial acylglycerols, phosphatides, metallic compounds, pigments, oxidation products, glycolipids, hydrocarbons, sterols, tocopherols, waxes, and phosphorous. In some embodiments, refinement removes certain minor components of the crude microbial oil with the least possible damage to the oil fraction (e.g., trans fatty acids, polymeric and oxidized triacylglycerols, etc.) and minimal losses of desirable constituents (e.g., tocopherols, tocotrienols, sterols, etc.). In some embodiments, processing parameters are adapted for retention of desirable minor components like tocopherols and tocotrienols and minimal production of unwanted trans fatty acids. See Gibon (2012) “Palm Oil and Palm Kernel Oil Refining and Fractionation Technology,” incorporated by reference herein in its entirety, for additional details of oil processing that are useful for the present microbial oils.

Common processing methods include physical refining, chemical refining, or a combination. In some embodiments, chemical refining comprises one or more of the following steps: degumming, neutralization, bleaching and deodorization. In some embodiments, physical refining comprises one or more of the following steps: degumming, bleaching, and steam-refining deodorization. While “physical refining” and “chemical refining,” as used herein and in the art, may refer to a general process of oil purification comprising multiple steps, possibly including bleaching and/or deodorizing, in the context of the present disclosure, the term “refined” as it relates to a microbial oil, e.g., a refined microbial oil, refers to a microbial oil from which one or more impurities or constituents have been removed other than odor and pigment. As such, stating that a microbial oil is refined does not indicate that the microbial oil has been deodorized and/or bleached. The term “RBD,” as used herein and as applied to a microbial oil, indicates that the microbial oil has been each of refined, bleached, and/or deodorized.

In some embodiments, in chemical refining, the free fatty acids and most of the phosphatides are removed during alkali neutralization. In some embodiments, the non-hydratable phosphatides are first activated with acid and further washed out together with the free fatty acids during alkali neutralization with caustic soda. In some embodiments, chemical refining comprises one or more steps of acid treatment, centrifugation, bleaching, deodorizing, and the like.

In some embodiments, during physical refining, phosphatides are removed by a specific degumming process and the free fatty acids are distilled during the steam refining/deodorization process. In some embodiments, the degumming process is dry degumming or wet acid degumming. In some embodiments, physical refining is employed when the acidity of the crude microbial oil is sufficiently high. In some embodiments, physical refining is employed for crude microbial oil with high initial free fatty acid (FFA) content and relatively low phosphatides.

In some embodiments, the microbial oil is deodorized. In some embodiments, the deodorization process comprises steam refining. In some embodiments, deodorization comprises vacuum steam stripping at elevated temperature during which free fatty acids and volatile odoriferous components are removed to obtain bland and odorless oil. Optimal deodorization parameters (temperature, vacuum, and amount of stripping gas) are determined by the type of oil and the selected refining process (chemical or physical refining) but also by the deodorizer design.

In some embodiments, the microbial oil is bleached. In some embodiments, the bleaching is performed through the use of bleaching earth, e.g., bleaching clays. In some embodiments, the bleaching method employed is the two stage co-current process, the counter-current process, or the Oehmi process. In some embodiments, the bleaching method is dry bleaching or wet bleaching. In some embodiments, bleaching is accomplished through heat bleaching. In some embodiments, bleaching and deodorizing occur concurrently.

In some embodiments, the microbial oil is refined, bleached, and/or deodorized.

In some embodiments, the microbial oil is not bleached or is only partially bleached. For example, in some embodiments, the microbial oil still retains pigments after processing. In some embodiments, the microbial oil comprises any one or more of the pigments referenced herein. Therefore, in some embodiments, the microbial oil is refined and deodorized, but not bleached or not fully bleached.

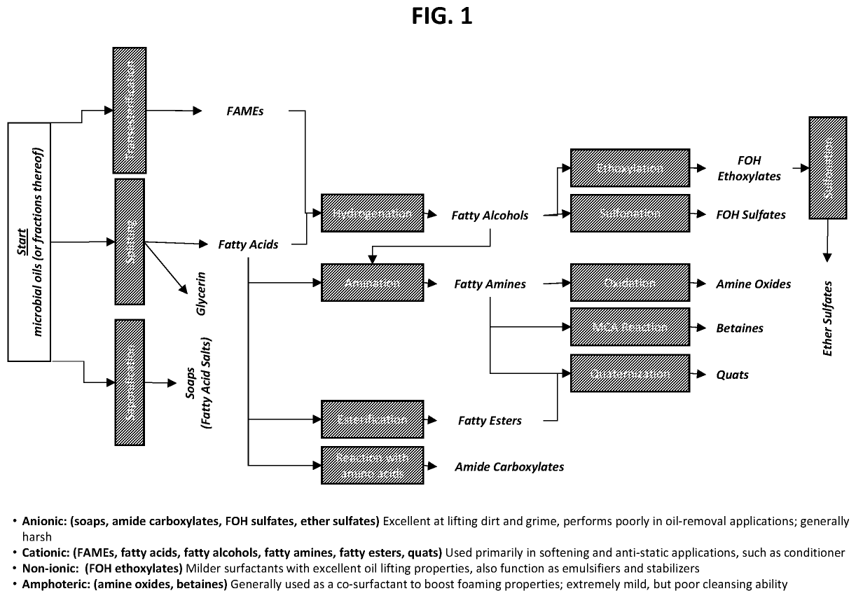

As shown in FIG. 1, the microbial oil may be processed or modified by a number of means to generate derivatives for use in personal care compositions. For example, as shown in FIG. 1, the microbial oil or fractions thereof may be modified by transesterification to produce FAMEs, or split (hydrolysis) to produce fatty acids and glycerin. These FAMEs and fatty acids may be subsequently hydrogenated to produce fatty alcohols. The fatty alcohols may undergo ethoxylation and/or sulfonation to produce fatty alcohol ethoxylates, fatty alcohol sulfates, or ether sulfates. The fatty acids may also be modified by amination, esterification, and reactions with amino acids to produce fatty amines, fatty esters, and amide carboxylates respectively. Fatty amines may further be modified by oxidation, monochloroacetic acid (MCA) reaction, and quaternization to produce amine oxides, betaines, and quats respectively. The microbial oil or fraction thereof may also be modified by saponification to produce fatty acid salts. Any of these derivatives and intermediate products may be used in personal care compositions. Thus, in some embodiments, the microbial oil is processed and/or modified via one or more of fractionation, hydrogenation, hydrolysis, distillation, saponification, esterification, interesterification, transesterification, amination, ethoxylation, sulfonation, oxidation, quaternization, MCA reaction, and/or reaction with amino acids.

### Fractionation

Fractionation of is another means of processing the microbial oil described herein for use in personal care compositions. Fractionation may be used to physically separate room temperature oil into saturated and unsaturated components. The melting points of full oil mixtures and their saturated/unsaturated components differ. Hydrophilization makes use of surface active agents (surfactants) that dissolve solidified fatty crystals and emulsify liquid oils. By centrifuging this hydrophilized suspension, fats can be separated into different fractions based on saturation.

In some embodiments, the microbial oil is fractionable. In some embodiments, the microbial oil is fractionable into two or more fractions. In some embodiments, the microbial oil is fractionable into more than two fractions. In some embodiments, the microbial oil is fractionable into two fractions, which may then be further fractionated.

In some embodiments, the microbial oil is fractionable into two fractions. In some embodiments, the two fractions are microbial olein and microbial stearin. In some embodiments, each fraction comprises at least 10% of the microbial oil's original mass. In some embodiments, the iodine value (IV) of the fractions differs by at least 10. In some embodiments, the iodine value of the fractions differs by at least 20. In some embodiments, the iodine value of the fractions differs by at least 30.

In some embodiments, the microbial oil is fractionated. In some embodiments, fractionation is carried out in multiple stages, resulting in fractions appropriate for different downstream indications. In some embodiments, the microbial oil is fractionated via dry fractionation. In some embodiments, the microbial oil is fractionated via wet fractionation. In some embodiments, the microbial oil is fractionated via solvent/detergent fractionation.

### Hydrolysis

Hydrolysis is the process whereby triglycerides in fats and oils are split (“fat splitting” or “oil splitting”) into glycerol and fatty acids. It is usually carried out using great amounts of high-pressure steam (“steam hydrolysis”) but may also be performed using catalysts (for example, the tungstated zirconia and solid acid composite SAC-13 (Hydrolysis of Triglycerides Using Solid Acid Catalysts, Ngaosuwan, K, et al., Ind. Eng. Chem. Res., 2009 48 (10), 4757-4767)). The reaction proceeds in a step-wise fashion wherein fatty acids on triglycerides are displaced one at time, generating diglycerides, then monoglycerides, and finally free fatty acids and glycerin.

In some embodiments, the microbial oil is split into free fatty acids and glycerol. In some embodiments, the microbial oil is split by steam hydrolysis. In some embodiments, the free fatty acids are further purified and/or separated into fractions through distillation or fractionation. In some embodiments, the resulting diglycerides, monoglycerides, free fatty acids, and glycerol are used in personal care compositions.

### Distillation

Distillation is a process whereby fatty acids and impurities are separated based on differences in boiling points. Fatty acids have a lower boiling point than impurities, such that the fatty acids may be vaporized, condensed, and collected, and the high-boiling impurities are left behind.

### Hydrogenation

Hydrogenation is the process whereby liquid fats are made solid or partially solid by adding hydrogen. The extra hydrogen converts the double bonds in unsaturated fats to single bonds, generating saturated fats. Unless the process is controlled, some fats may be partially hydrogenated and this leads to “trans fats”, so named due to the trans configuration of the molecule. In the U.S., artificial trans fats have been banned from food products, however hydrogenated fats may still be used in personal care compositions. Hydrogenated oils prevent the rancid odors caused by oxidation, thus increasing the shelf life of the product, and may also provide a thicker consistency.

The FAMES produced by transesterification may be hydrogenated to produce fatty alcohols. Fatty acids produced from hydrolysis may also be further modified via esterification to produce wax esters, which may then be hydrogenated to produce fatty alcohols. Direct hydrogenation of fatty acids is also possible and produces fatty alcohols. Thus, in some embodiments, the oil is derivatized to fatty alcohols. In some embodiments, fatty alcohols derived from an oleaginous yeast are used in a personal care item. In some embodiments, the fatty alcohols are further refined and/or distilled. In some embodiments, the fatty alcohols are further derivatized by ethoxylation and/or sulfonation. In some embodiments, the fatty alcohol derivative is an ethoxylated fatty alcohol. In some embodiments, the disclosure relates to a composition of matter comprising cetearyl alcohol derived from fatty alcohols produced by an oleaginous yeast.

In some embodiments, the fatty acids derived from the microbial oil are distilled. In some embodiments, the disclosure teaches methods of using free fatty acids from oleaginous microorganisms in personal care compositions. In some embodiments, the disclosure relates to a personal care composition comprising a fatty acid-ingredient derived from an oleaginous yeast. In some embodiments, the fatty acid-ingredients are selected from the group consisting of stearic acid, oleic acid, palmitic acid, and myristic acid.

### Amination

Fatty amines are another class of oleochemicals commonly derived from C12-C18 hydrocarbons from fatty acids. They are produced through the hydrogenation of fatty nitriles, which are themselves produced from a reaction between triglycerides, fatty acids, or fatty esters with ammonia and a catalyst. Fatty amines and their derivatives may be used, for example, in antistatic products, antimicrobial products, shampoos, conditioners, liquid cleansers, and oral care products.

Thus, in another embodiment, the fatty acids, triglycerides, and/or fatty esters derived from the microbial oil described herein may be used to produce fatty amines. In another embodiment, the disclosure relates to personal care compositions comprising fatty amines derived from a microbial oil.

### Saponification

Saponification is the process whereby triglycerides or free fatty acids used as feedstock are converted to fatty acids salts (soaps), glycerol, and free fatty acids in the presence of a base. The base may be for example, sodium hydroxide, which for example produces hard bar soaps, or potassium hydroxide, which for example produces softer bars or liquid soaps. Saponification may be achieved via a hot or cold process. The cold process uses the heat generated from the combination of the fatty acids in the melted oils and fats with sodium hydroxide (base). This process takes longer, and an additional curing phase is needed for the soap to harder. The hot process uses heat to speed up the saponification process, and generally no additional curing step is required before use of the soap.

Globally the soap bar industry is worth approximately US$186 billion. The most commonly used oil sources are vegetable oils, specifically palm and coconut oils, which contain shorter saturated fatty acids. These shorter chain saturated fatty acids increase the lathering profile, while longer saturated fatty acids contribute to the hardness of the soap. Unsaturated fatty acids provide moisture, conditioning, or skin nourishing properties. While animal fats may be used, vegetable oils generally produce higher quality soaps (Prieto Vidal N, et al., The Effects of Cold Saponification on the Unsaponified Fatty Acid Composition and Sensory Perception of Commercial Natural Herbal Soaps. Molecules. 2018; 23 (9): 2356).

In some embodiments, the microbial oil, free fatty acids, and/or triglycerides are used as feedstock in a saponification reaction to produce fatty acid salts, glycerol, and/or free fatty acids. In some embodiments, these fatty acid salts, glycerol, and/or free fatty acids are used in a personal care composition. In some embodiments, the personal care composition is soap.

Sodium stearate is produced by saponification of stearic acid, and it one of the most commonly used commercial surfactants in soap. It is also found in solid deodorants, rubbers, latex paints, and inks. In one embodiment, the disclosure relates to a sodium stearate produced from stearic acid, wherein the stearic acid is produced by an oleaginous yeast. In another embodiment, the disclosure relates to products and compositions comprising a sodium stearate derived from an oleaginous yeast.

### Esterification

Esterification is the general name for a reaction that generates esters, a compound derived from an acid. Esterification of fatty acids can generate nonionic surfactants (see for example, Li X., et al., Fatty acid ester surfactants derived from raffinose: Synthesis, characterization and structure-property profiles, 2019, J. of Colloid and Interface Science, Vol. 556(15); 616-627). For example, glycerol esters can be used as emulsifiers, dispersants, and solubilizing agents.

Many esters have fruit-like odors and occur naturally in essential oils of plants, and may be used in fragrances to mimic those odors. In some embodiments, the microbial oil is derivatized to esters. In another embodiment, esters derived from an oleaginous yeast are used in a personal care composition. In some embodiments, the esters are used as a fragrance in a personal care composition.

In some embodiments, the microbial oil is modified via interesterification. In some embodiments, the interesterification is enzymatic. In some embodiments, the interesterification is chemical. In some embodiments, the microbial oil is modified via transesterification. In some embodiments, the oil is derivatized to fatty acid methyl esters (FAMEs). Methyl esters may be used in personal care items, for examples perfumes and soap, or they may be a carrier for an active ingredient, an emollient, or viscosity regulator. Thus, in another embodiment, the FAMEs derived from oleaginous yeast are used in a composition of matter.

produce methyl esters, which may then be hydrogenated to produce fatty alcohols. Direct hydrogenation of fatty acids is also possible and produces fatty alcohols. Thus, in some embodiments, the oil is derivatized to fatty alcohols. In some embodiments, fatty alcohols derived from an oleaginous yeast are used in a personal care item. In some embodiments, the disclosure relates to a composition of matter comprising cetearyl alcohol derived from fatty alcohols produced by an oleaginous yeast.

### Surfactants

An embodiment of the present disclosure relates to personal care compositions comprising a microbial oil, and/or derivative thereof, wherein the derivative functions as a surfactant in a personal care composition. In another embodiment, the present disclosure relates to personal care compositions comprising a microbial oil, and/or derivative thereof, and a surfactant. In some embodiments, the surfactant is an emulsifier, detergent, wetting agent, foaming agent, thickening agent, or emollient. In personal care items, emollients provide skin softening or soothing properties, and may comprise, for example, triglycerides, hydrocarbons, silicons, and esters.

Surfactants are a broad category of compounds that lower the surface tension between two liquids, for example oil and water, between a gas and a liquid, or between a liquid and a solid. Depending on the compound, they may act as an emulsifier, emollient, detergent, wetting agent, foaming agent, thickening agent, pearlescent, solubilizer, conditioning agent, co-surfactant, or dispersant. In some instances, they can act as an anti-microbial agent and/or a preservative. They can be classified by their head group as either non-ionic (neutral), anionic (negatively charged), cationic (positively charged), or amphoteric (both positive and negative charges).

Examples of emollients, emulsifiers, and other surfactants are shown below in Tables 3a and 3b. The lists of surfactants in Tables 3a and 3b below illustrate some, but not the only or exclusive, example surfactants. It is intended that the surfactants disclosed herein are to be considered illustrative rather than limiting. Some of these surfactants may be produced using the microbial oils described herein, for example, those listed in Tables 3a and 3b below with an asterisk (for example, stearyl alcohol, ethylhexyl palmitate, etc.). As will be understood by one skilled in the art, the microorganisms described herein may be tailored to produce more less of a particular lipid, for example, C12 (lauric acid). Thus, while derivatives of lauric acid are not marked with an asterisk below, they are within the scope of a microbial oil derivative. Other surfactants listed may be used as additional ingredients in compositions comprising microbial oil, triglycerides, diglycerides, monoglycerides, free fatty acids, fatty acid salts, glycerol, esters, and/or fatty alcohols derived from an oleaginous yeast. O/W means oil-in-water; W/O means water-in-oil.

TABLE 3a
Examples of surfactants

| Name | Function | Example use |
|---|---|---|
| Lauryl Laurate | Emollient. Improves slip of | Lotions, serums |
| | formulation and helps increase | |
| | stability of formulation | |
| Cocoamphocarboxyglycinate | Amphoteric surfactant, foaming | Cosmetics, cleansers, |
| | agent | hair care |
| Stearyl alcohol* | Emollient, thickener | Cosmetics, perfumes, |
| | | deodorants, |
| | | antiperspirants |
| Sodium cocoyl isethionate | Mild surfactant, emulsifier, | Liquid and bar |
| | foaming agent | shampoos, body wash, |
| | | bath bombs, cleanser, |
| | | baby products |
| Sodium lauryl sulphoacetate | Foaming agent, wetting agent | Shampoo, cleansers, hair |
| | | treatments, bubble bath, |
| | | bath oils/salts/bombs |
| Sodium methyl cocoyl | High-foaming agent, mild | Cleansers, shampoo, |
| taurate | surfactant | facial masks and scrubs, |
| | | anti-acne treatments |
| Ethylhexyl Palmitate* | Emollient, pigment wetting agent, | Lotions, conditioner, |
| | thickening agent, skin conditioner | sunscreen, cosmetics |
| Ethylhexyl Isononanoate | Emollient and sensory ingredient. | Lotions, hair styling, |
| | Enhances product feel, non-greasy | aerosol spray products, |
| | | antiperspirants, |
| | | deodorants |
| Ethylhexyl Stearate* | Thickening agent, emollient, skin | Cosmetics, lotions, |
| | conditioning agent, wetting agent | sunscreen, conditioner |
| | for pigments and powders | |
| Ethylhexyl Adipate | Light emollient, glossing agent, | Lotions, sunscreen |
| | film formation aid | |
| Ethylhexyl Ethylhexanoate | Emollient | Lotions, sunscreen |
| Ethylhexyl cocoate | Emollient, non-comedogenic | Lip gloss, lipstick, |
| | | antiperspirants, |
| | | deodorants, face and |
| | | body lotion, sunscreen |
| Octyldodecyl Stearate* | Emollient, thickener | Skin care, cleansers, |
| | | cosmetics |
| Decyl polyglucose | Mild non-ionic surfactant | Body wash, shampoo |
| Decyl glucoside | Mild non-ionic surfactant | Cosmetics, baby |
| | | shampoo, sensitive skin |
| | | products |
| Isononyl Isononanoate | Emollient and Lubricating agent. | Lotions, sunscreen, hair |
| | Reduces greasiness | care, anti-stretch mark |
| | | products |
| Capric/Caprylic | Emollient. Form a protective | Lotions, sunscreen, |
| Triglycerides | barrier around the skin to hold in | conditioner, firming body |
| | moisture | lotions, protective day |
| | | creams |
| Sorbitan Oleate* | Nonionic O/W co-emulsifier, W/O | Lotions, sunscreen, |
| | emulsifier | conditioner |
| Sorbitan Trioleate* | Nonionic O/W co-emulsifier, W/O | Lotions, sunscreen, |
| | emulsifier | conditioner |
| Sorbitan Isostearate* | Nonionic emulsifier and dispersing | Lotions, sunscreen |
| | agent | |
| Sorbitan Laurate | Nonionic O/W co-emulsifier, W/O | Lotions, sunscreen, |
| | emulsifier | conditioner |
| Sorbitan Oleate* | Nonionic O/W co-emulsifier, W/O | Lotions, sunscreen, |
| | emulsifier | conditioner |
| Sorbitan Stearate* | Nonionic O/W co-emulsifier, W/O | Lotions, sunscreen, |
| | emulsifier | conditioner |
| Sorbitan Sesquioleate | Emulsifier | Lotions, sunscreen, |
| | | conditioner |
| Sorbitan Trioleate* | Nonionic O/W co-emulsifier, W/O | Lotions, sunscreen, |
| | emulsifier | conditioner |
| Isotridecyl Stearate* | Emulsifier | Lotions |
| Sucrose cocoate | Mild surfactant, emulsifier, | Hair products, cleansers, |
| | antistatic | lotions, creams |
| Beheneth-10 | O/W emulsifier. Especially suited | Lotions, conditioner |
| | for low solids formulations | |
| Glyceryl Stearate* | O/W emulsifier. | Lotions, conditioner |
| PEG-100 Stearate* | O/W emulsifier. | Lotions, conditioner |
| Laureth-9 | O/W emulsifier for the formulation | Lotions |
| | of skin soothing creams with low | |
| | melting point | |
| Cetoleth-20 | O/W emulsifier for formulation of | Lotions, sunscreen |
| | micro-emulsions | |
| Laureth-4 | O/W emulsifier, well suited for oil | Lotions, body wash |
| | containing body wash | |
| Polysorbate 20 | Solubilizer for essential oils | Body wash, shampoo |
| Polysorbate 40* | Emulsifier and stabilizer | Cleansers, shampoo, |
| | | makeup |
| Polysorbate 60* | Emulsifier, solubilizer | Cleansers, shampoo, |
| | | makeup |
| Polysorbate 80* | O/W emulsifier, oil solubilizer | Lotions, shampoo, body |
| | | wash |
| Ceteareth-20* | O/W emulsifier | Lotions |
| Steareth-2* | O/W emulsifier | Lotions |
| Steareth-20* | O/W emulsifier | Lotions |
| Steareth-21* | O/W emulsifier | Lotions |
| PPG-1 Trideceth-13 | Fragrance Solubilizer | Lotions, body wash |
| PEG-4 Dilaurate | Emulsifier | Lotions |
| PEG-4 Dioleate* | Emulsifier | Lotions |
| PEG-4 Laurate | Emulsifier, emollient | Lotions, sunscreen |
| PEG-4 Oleate* | Emulsifier, emollient | Lotions, sunscreen |
| PEG-4 Stearate* | Emulsifier, emollient | Lotions, sunscreen |
| C12-15 Pareth-12 | Fragrance solubilizer for surfactant | Body wash, shampoo |
| | formulations that doesn't affect the | |
| | lathering or thickening of the | |
| | finished formulations | |
| PEG-6 Laurate | Emulsifier, emollient | Lotions, sunscreen |
| PEG-6 Oleate* | Emulsifier, emollient | Lotions, sunscreen |
| PEG-6 Stearate* | Emulsifier, emollient | Lotions, sunscreen |
| PEG-7 Glyceryl cocoate | Emulsifier, emollient, thickener, | Liquid soaps, body |
| | foam booster, conditioner z | washes, shampoo, face |
| | | wash, lotions, bubble |
| | | baths, cosmetics |
| PEG-8 Dilaurate | Emulsifier, emollient | Lotions, sunscreen |
| PEG-8 Dioleate* | Emulsifier, emollient | Lotions, sunscreen |
| PEG-8 Laurate | Emulsifier, emollient | Lotions, sunscreen |
| PEG-8 Oleate* | Primary emulsifier, emollient, | Lotions, sunscreen |
| | nonionic surfactant | |
| PEG-8 Stearate* | Emulsifier, emollient | Lotions, sunscreen |
| PEG-12 Dilaurate | Emulsifier, emollient | Lotions, sunscreen |
| PEG-12 Dioleate* | Primary emulsifier, emollient, | Lotions, sunscreen |
| | nonionic surfactant | |
| PEG-12 Distearate* | Emulsifier, emollient | Lotions, sunscreen |
| PEG-12 Laurate | Emulsifier, emollient | Lotions, sunscreen |
| PEG-12 Monooleate* | Emulsifier, emollient | Lotions, sunscreen |
| PEG-12 Stearate* | Emulsifier, emollient | Lotions, sunscreen |
| PEG-60 Almond glyceride | Emulsifier, emollient | Shampoo, hair styling |
| | | products, toners, body |
| | | wash/cleanser, makeup |
| | | remover |
| PEG-150 Distearate* | Emulsifier, emollient | Lotions, sunscreen |

Additional surfactants and examples of their functions and use are shown below in Table 3b.

TABLE 3b
Additional surfactants

| Name | Function | Example use |
|---|---|---|
| Cocamidopropyl Betaine | Secondary amphoteric | Shampoo, body wash |
| | surfactant, helps improve | |
| | richness of lather of shampoo & | |
| | body wash formulations. | |
| | Increases the mildness of | |
| | formulations | |
| TEA-Lauraminopropionate | Amphoteric surfactant with low | Lotions, body and face |
| | foaming but good wetting | wash |
| | properties. Can be used in a | |
| | large range of pHs | |
| MIPA-Laureth Sulfate Laureth-4 | Anionic surfactant blend to aid | Shower and bath oils |
| Cocamide DEA | in the foaming of shower and | |
| | bath oils. Used in anhydrous | |
| | formulations | |
| Disodium Laureth | Anionic surfactant blend with | Sulfate-free |
| Sulfosuccinate | foaming properties and mild to | formulations, face |
| Sodium Lauryl Sulfoacetate | the skin. | wash, shampoo, baby |
| | | wash and baby |
| | | shampoo |
| MIPA-Laureth Sulfate | Anionic surfactant blend to aid | Shower and bath oils |
| Laureth-4 | in the foaming of shower and | |
| | bath oils. Used in anhydrous | |
| | formulations | |
| Sodium Lauroyl Sarcosinate | Anionic surfactant blend for | sulfate -free |
| Sodium Lauryl Sulfoacetate | shampoo & body wash | formulations, |
| | formulations. Provides mildness, | shampoo, body wash |
| | foaming properties & viscosity | |
| | control | |
| PEG-90 Glyceryl Isostearate* | Thickening agent for body | Shampoo, body wash |
| Laureth-2 | washes and shampoos. Improves | |
| | foaming ability and stability | |
| Sodium Cocoyl Glutamate | Mild anionic surfactant used to | Shampoo, body wash |
| | decrease irritation from | |
| | SLES/SLS and acts as an | |
| | emulsifier for lipids | |
| Sodium Lauroyl Glutamate | Mild anionic surfactant | Shampoo, body wash, |
| | compatible with other anionic, | baby wash & baby |
| | nonionic, and amphoteric | shampoo |
| | surfactants. Improves skin | |
| | compatibility in such | |
| | formulations | |
| Disodium Cocoyl Glutamate | Mild anionic surfactant with | Shampoo, baby wash |
| | moderate foaming power. | & baby shampoo |
| Glyceryl Stearate * | O/W emulsifier with | Lotions |
| Cetearyl Alcohol* | moisturizing effect | |
| Stearic Acid* | | |
| Sodium Lauroyl Glutamate | | |
| Sodium Lauroyl Sarcosinate | Very mild surfactant suitable for | Shampoo, face wash, |
| | sensitive skin. Improves foam | body wash; Baby |
| | and thickening properties of | wash & baby shampoo |
| | formulation | |
| Sodium Cocoyl Sarcosinate | Very mild surfactant suitable for | Shampoo, face wash, |
| | sensitive skin. Suitable for baby | body wash; Baby |
| | care products. Improves foam & | wash & baby shampoo |
| | thickening of formulation | |
| Sodium PCA | Humectant, unpreserved solution | Shampoo, conditioner, |
| | of Sodium PCA in glycerol and | lotions |
| | water | |
| Sodium Cocoyl Hydrolyzed | Mild surfactant that improves | Shampoo, body wash, |
| Wheat Protein | skin compatibility of surfactant | baby wash |
| | formulations | |
| Lauryl PCA | Skin conditioning agent & | Lotions, conditioner |
| | moisturizer. Melting point | |
| | slightly under body temperature, | |
| | which allows product to melt on | |
| | the skin | |
| Octyldodecyl PCA | Skin conditioning agent that | Lotions, body wash |
| | spreads easily and absorbs | |
| | quickly. | |
| Isostearyl PCA | Specialty moisturizing ester that | Body wash, face wash |
| | spreads easily and absorbs | |
| | quickly. Provides structure to the | |
| | foam of rinse off formulations | |
| Disodium Laureth | Anionic surfactant, improves | Shampoo, body wash |
| Sulfosuccinate | mildness of formulation | |
| Sodium Laureth Sulfate (SLS) | Anionic detergent | Shampoo, body wash, |
| | | shower wash/gel, |
| | | cleansers, hand soap |
| Sodium Lauryl Sulfate (SLES) | High foaming anionic surfactant | Shampoo, body wash, |
| | with antimicrobial properties | toothpaste, mouth rinse, |
| | | hand soap, hand |
| | | sanitizers, disinfectants |
| Ammonium Lauryl Sulfate | High foaming anionic surfactant | Shampoo, body wash |
| Magnesium Lauryl Sulfate | Mild anionic surfactant that is | Shampoo, body wash, |
| | skin compatible | baby wash |

### Cleaning Agents

An embodiment of the present disclosure relates to personal care compositions comprising a microbial oil, or derivative thereof, and a cleaning agent.

A cleaning agent is any substance used to remove dirt, dust, stains, and/or odor. They may also be classified as a disinfectant or anti-microbial, and may also be classified as a surfactant. Cleaning agents may comprise liquids, powders, sprays, or granules, for example, pumice soapstone, and talc. In some embodiments, the cleaning agent is an alkaline solution, acidic solution, neutral solution, degreaser, or scouring agent. In some embodiments, the alkaline solution is sodium hydroxide (also known as lye) or potassium hydroxide.

### Polymers

An embodiment of the present disclosure relates to personal care compositions comprising a microbial oil, or derivative thereof, and a polymer.

Polymers are a broad category of substances composed of the same, or similar, repeating subunits (monomers), and may be natural or synthetic. Polymers are routinely used in many personal care and cosmetic products. In general, those polymers used in personal care items are liquid (as opposed to solid, for example - plastic). Types of personal care products containing polymers which may be used in the compositions described herein include, but are not limited to, lotions, creams, hair care products, and cosmetics.

Depending on the polymer, it may act as a thickener, structuring agent, emulsifier, emollient, moisturizer, delivery (“carrier”) system (for example, to deliver an active ingredient), film former (for example, nail polish), or to waterproof (for example waterproof make-up and sunscreen). As will be understood by one skilled in the art, the choice of polymer in a personal care or cosmetic product depends on the application, formulation, and desired result.

Examples of polymers with may be used in the compositions described herein include hyaluronic acid, collagen, protein, starch, xanthan or guar gum, carrageenan, alginates, polysaccharides, pectin, gelatin, agar, and cellulose derivatives, corn starch, natural and synthetic waxes (for example rice bran wax), lanolin, long-chain fatty alcohols, triglycerides, poly-alpha-olefin, glycol stearates, polyvinyl pyrrolidone, acetate, polyvinylamides, polyacrylates, polymethacrylates, polyurethanes, silicones, polyquaternium-6, polyquaternium-7, and polyquaternium-11.

In another embodiment, the polymer used in the composition is derived from an oleaginous yeast. In some embodiments, the polymer is a long-chain fatty alcohol, triglyceride, or glycol stearate derived from an oleaginous yeast.

### Luxury Soft Oils

In another embodiment, the disclosure relates to personal care compositions comprising a microbial oil, or derivative thereof, and a luxury soft oil.

Examples of luxury soft oils include, but are not limited to, argan oil, jojoba oil, meadowfoam seed oil, black seed oil, evening primrose oil, walnut oil, wheat germ oil, hemp oil, rosehip oil, and pumpkin seed oil,

### Essential Oils & Fragrance Oil

In another embodiment, the disclosure relates to personal care compositions comprising a microbial oil, or derivative thereof, and an essential oil.

Examples of essential oils that may be used in the compositions described herein include for example, amyris, bergamot, black pepper, cardamom, cedarwood, chamomile, clary sage, eucalyptus, geranium, ginger, grapefruit, juniper, lavender, lemongrass, lemon, lime, may chang, neroli, nutmeg, palmarosa, patchouli, peppermint, petitgrain, rose, rosemary, rosewood, sandalwood, scots pine, spearmint, sweet marjoram, orange, tea tree, vetiver, and ylang ylang.

In another embodiment, the compositions described herein may also comprise a fragrance oil. As will be understood by one skilled in the art, there are hundreds of fragrance oils, any one of which may be used with the compositions described herein.

### Antioxidants

In personal care items, antioxidants can prevent free radicals from oxidizing other ingredients, such as proteins, sugars, and lipids. For example, in soap, oxidation of the double bonds of lipids can produce shorter chain fatty acids, aldehydes, and ketones, which yield odors and discoloration. Antioxidants in personal care compositions may increase the shelf life of that product. Antioxidants can also provide benefits to the end user of the personal care item, for example, antioxidants may help to reduce or minimize sun spots, increase skin's radiance, and prevent wrinkles.

Examples of antioxidants include, but are not limited to, vitamin E, coenzyme Q10, idebenone, lycopene, vitamin C, green tea, silymarin, Resveratrol, grape seed, pomegranate extracts, coffee bean extracts, genistein, pycnogenol, tetrahydrodiferuloylmethane, tocopherol (for example Covi-ox® T 50 C), and niacinamide. As will be understood by one skilled in the art, an antioxidant may also be classified as a biologically active ingredient.

In another embodiment, the compositions described herein may also comprise an antioxidant.

### Biologically Active Ingredients

“Biologically active ingredients” or “active ingredients” or “biologically active compounds” are those ingredients which have a physiological effect. In another embodiment, the disclosure relates to personal care compositions comprising a microbial oil, and/or derivative thereof, and a biologically active ingredient. In some embodiments, the biologically active ingredient is an antibiotic, antimicrobial, anti-inflammatory, antioxidant, mineral, or inorganic compound derived from a mineral. In some embodiments, the composition is a cosmeceutical.

Examples of active ingredients that may be used with the compositions described herein may include, but are not limited to, zinc oxide, titanium dioxide, avobenzone, oxybenzone, vitamin A/retinoids/retinol, bakuchiol, vitamin C, vitamin E, hyaluronic acid, kojic acid, AHAs, BHA, hydroquinone, salicylic acid, benzoyl peroxide, azelaic acid, antibiotics, azelaic acid, sulfur, steroids, urea, lactic acid, anthralin, tacrolimus, and pimecrolimus.

In some embodiments, the composition is a lotion. In some embodiments, the composition is a sunscreen. In some embodiments, the composition is a wrinkle cream. In some embodiments, the composition is a deodorant. In some embodiments, the composition is an acne treatment. In some embodiments, the composition is an eczema or psoriasis treatment.

### Characteristics Similar to Other Ingredients Used in Personal Care Compositions

The microbial oil and/or derivatives thereof described herein may also function as a replacement for other ingredients in personal care compositions. For example, beeswax is a common component in oil-based balms. A wax-ester blend derived from an oleaginous microorganism may be used in place of beeswax in personal care compositions. Fractionated microbial olein and fractionated microbial stearin may replace rice bran oil and shea butter, respectively, or may serve as a replacement to agricultural palm olein and palm stearin (and traditional derivatives thereof). Fractioned olein may also replace vegetable oils high in oleic acids, such as tea seed oil. As will be understood by one skilled in the art, the oleaginous microorganisms described herein may be tailored to produce more or less of a particular hydrocarbon, for example C12 (lauric acid). Lauric acid and derivatives thereof are also used in personal care compositions. The microbial oil and/or derivatives thereof may further function as a biologically active ingredient.

Thus, in another embodiment, the disclosure relates to personal care compositions comprising microbial oil and/or derivative thereof, wherein the microbial oil or derivative thereof functions as a biologically active ingredient.

The present description is made with reference to the accompanying drawings and Examples, in which various example embodiments are shown. However, many different example embodiments may be used, and thus the description should not be construed as limited to the example embodiments set forth herein. Rather, these example embodiments are provided so that this disclosure will be thorough and complete. Various modifications to the exemplary embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

Although the disclosure may not expressly disclose that some embodiments or features described herein may be combined with other embodiments or features described herein, this disclosure should be read to describe any such combinations that would be practicable by one of ordinary skill in the art. Unless otherwise indicated herein, the term “include” shall mean “include, without limitation,” and the term “or” shall mean non-exclusive “or” in the manner of “and/or.”

Those skilled in the art will recognize that, in some embodiments, some of the operations described herein may be performed by human implementation, or through a combination of automated and manual means. When an operation is not fully automated, appropriate components of embodiments of the disclosure may, for example, receive the results of human performance of the operations rather than generate results through its own operational capabilities.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world, or that they disclose essential matter.

### EXAMPLES

### Example 1: Rhodosporidium toruloides Fermentation on Industrial Fermentation Waste Stream Feedstock

Exemplary feedstocks of corn stillage syrup, corn thin stillage, corn whole stillage, and corn stillage pre-blend were used in the fermentation of an exemplary oleaginous yeast, Rhodosporidium toruloides.

### Feedstock Preparation

Corn stillage syrup has a high viscosity and requires centrifugation and/or dilution prior to use as a feedstock. Five preparations of the feedstock were made. Preparation A comprised a 10% v/v dilution of the feedstock with water (10% syrup, 90% water), spun down at 5000 g for 10 min to remove insoluble components. Preparation B comprised the feedstock spun down at 5000 g for min to remove insoluble components without dilution. Preparation C comprised a 10% dilution of the feedstock with water (10% syrup, 90% water). Preparation D comprised a 20% dilution of the feedstock with water (20% syrup, 80% water). Preparation E comprised a 30% dilution of the feedstock with water (30% syrup, 70% water). These preparations were compared to a theoretically optimized medium comprising a C/N ratio of 100 (control preparation).

### Culturing R. toruloides

100 mL of each preparation was autoclaved in a 250 mL flask and inoculated to OD600=1 with precultures of R. toruloides cultivated overnight in YPS (10 g/L yeast extract, 20 g/L peptone, g/L sucrose). The cultures were shaken at 200 rpm, 30° C. for 6 to 9 days, and the cells were harvested by centrifugation.

### Determination of Biomass and Lipid Content

The harvested cells were dried in a vacuum oven at 60° C. until their mass was stable. The cells were weighed and lipids were subsequently extracted by acid lysis followed by chloroform:methanol separation. In short, 400 mg of powdered biomass were mixed with 3.2 mL of 4 M hydrochloric acid and incubated at 55° C. for 2h. 8 mL of chloroform:methanol 1:1 were added. The solution was shaken vigorously for 3h and centrifuged at 6000 g for 15 min. The chloroform layer was separated and 4 mL of pure chloroform were added. The solution was shaken vigorously for 30 min, spun down again, and the chloroform layer was separated. The samples in chloroform were dried under nitrogen stream until mass was constant and mass was recorded.

Corrections were applied to the measured lipid titers in order to account for lipids already present in the feedstock preparations. Lipids from the “blank” preparations were extracted as described above for the cultures, and the amount of lipids in the blank was subtracted from the amount of lipids in the cultures in order to get the actual amount of lipids produced by R. toruloides. This process provides a low conservative estimate of the lipids produced by the microorganisms, as the yeast consume some portion of the lipids present in the feedstock. For Preparation A and Preparation B, the lipids were separated via centrifugation of the broth prior to inoculation, so no correction was applied.

### Results

Various preparations of the feedstock were able to support oleaginous microbial growth and lipid production. The feedstock contains high concentrations of glycerol, making it viscous and decreasing oxygen transfer. As such, because of the high viscosity of the feedstock, it is impractical for use as is in a shaken vessel, which is why preparation B produced lower biomass production than the other preparations. However, dilutions of the feedstock and clarification by centrifugation (removal of solids) allow for good biomass and lipid production. Increased concentrations of feedstock lead to increasing biomass production and lipid titer (preparations C, D, and E), until viscosity limits oxygen and nutrient transfer (preparation B). Preparation E performed as well as the optimized control preparation. Further, it was shown that lipid content can be optimized by adjusting the time period of fermentation.

### Example 2: Media Formulation, Fermentation, and Lipid Extraction

### Media Formulation

For the following examples, fermentation feedstocks were acquired in 4 separate formulations—i.e., as whole stillage, thin stillage, clarified stillage, and syrup. Fermentation media formulations were optionally diluted in deionized water at various fractions (a “10% feedstock” medium indicates a 1:9 ratio of feedstock fraction to water). Feedstocks were optionally fractionated into supernatant and solid fractions via centrifugation.

### Fermentation

To prepare inocula, yeast strains were propagated at 30° C., 200rpm, for 28 hours in yeast extract-peptone-dextrose (YPD) medium composed of 10 g/L yeast extract, 20 g/L peptone, and 20 g/L dextrose. Cultures were washed of residual nutrients before inoculating 100 mL of the exemplary feedstock to a starting OD600 of 1.0.

Fermentations were run in 250 mL baffled flasks within an orbital shaker incubator at 200 rpm and 30° C. During harvest, cultures were centrifuged at 4700 g for 10 minutes to pellet, resuspended in 20 mL deionized water, and centrifuged again to yield a washed, wet cell pellet.

### Insoluble Matter Correction and Solvent Pre-Wash Procedure Before Lipid Extraction

Some exemplary feedstocks formulated from post-fermentation waste streams contain insoluble matter that needs to be removed or quantified to result in accurate microbial biomass and lipid content calculations. To correct for this content within the feedstock itself, blank cultures were prepared and collected to assess the carryover weight of insoluble matter in the exemplary feedstock. For feedstocks formulated from corn thin stillage, the biomass was able to be separated from the insoluble matter, such that no correction was required. For feedstocks formulated from post-fermentation media clarified supernatants after centrifugation, the feedstock did not comprise insoluble matter, and no correction was necessary.

For all feedstock formulations, the extraction of native lipids was performed on the wet cell pellet/feedstock mixture using a solvent pre-wash of 20 mL chloroform/methanol mixture (2:1 v/v). The pellet and pre-wash mixture was then incubated in a 50° C. water bath for 10 minutes. Next, it was incubated in a 50° C. shaker at 400 rpm for 45 minutes. Samples were centrifuged at 4700 g for 10 minutes to induce phase separation. Chloroform and methanol were carefully decanted. The insoluble matter of the diluted corn stillage syrup feedstock could not be separated from the biomass to obtain a pure wet cell pellet, but the biomass could be separated from the thin stillage insoluble matter using 250 g/L sorbitol for a density gradient. Collected and washed cultures were resuspended in 45 mL 250 g/L sorbitol then centrifuged at 4700×g for 10 minutes. The top layer that formed was the desired biomass, whereas the insoluble matter collected at the bottom. The biomass layer was isolated and washed in 45 mL deionized water to obtain the wet cell pellet.

### Lipid Extraction

Biomass was dried to a constant mass in a vacuum oven. Dry cell weight (DCW) was then measured, with correction for insoluble matter as needed. Dried biomass was lysed with 8 mL 4M HCl at 55° C., mild agitation for two hours and extracted with 8 mL chloroform/methanol mixture (2:1 v/v) at room temperature, 350 rpm for three hours. The mixture was centrifuged at 4700 g for 10 minutes. The lower layer of chloroform with extracted lipids was isolated and re-extracted using 4 mL chloroform at room temperature, 350 rpm for 30 minutes. Chloroform was evaporated to finalize the lipid extraction. Oil titer was then calculated, with correction for contributions from insoluble matter as needed. Lipid content was determined by dividing oil titer by dry cell weight.

### Example 3: Fermentation of Oleaginous Microorganisms on Exemplary Feedstock

### Feedstock

For the purposes of this example, the exemplary feedstock employed was a 30% corn stillage syrup-based feedstock, comprising 30% v/v corn stillage syrup, with insoluble components removed via centrifugation, diluted in deionized water.

### Microorganisms

Four strains of oleaginous microorganisms were selected to investigate the potential of the exemplary feedstock to support the growth of oleaginous microorganisms: R. toruloides strain A, R. toruloides strain B, Y. hpolytica strain polg, and L. starkeyi strain CBS 1807. As a control, a canonical non-oleaginous yeast, P. pastoris strain X33, was included for comparison.

### Batch Fermentation Results

Fermentations were run in batch format for 6 days. Dry cell weight, oil titer and lipid content were evaluated as described in Example 5.

The feedstock supported cell growth for all of the tested yeast strains. The two strains of R. toruloides performed the best in terms of oil titer and lipid content.

### Batch Fermentation Results with Glycerol Supplement

50 g/L glycerol was added to the feedstock and fermentations were again run for 6 days. Dry cell weight and oil titer were evaluated and compared to the results of the batch fermentation. Surprisingly, both strains of R. toruloides dramatically increased growth and oil production with added glycerol, while Y. lipolytica and L. starkeyi experienced significantly less growth and only marginally improved oil titer.

### Example 4: Fed Batch Fermentation With R. toruloides

A strain of R. toruloides was tested in a fed-batch fermentation format on two different exemplary feedstocks of the disclosure: 30% stillage and 40% stillage. The 30% and 40% stillage feedstocks were formulated with 30% and 40% corn stillage syrup, respectively, diluted in deionized water. The strain was also grown on defined media as a control. The carbon source for this fed batch fermentation was pure glycerol. The cultures were periodically sampled to measure residual glycerol concentration (via HPLC) and then fed with a bolus of concentrated glycerol (800 g/L) to replenish carbon to 60 g/L.

The fermentations were run for 7 days (˜148 hrs). Total target glycerol (batch+feed) was 281.5 g/L. The cells were then evaluated for DCW, oil titer, lipid content, productivity, and yield. Productivity was calculated by taking the oil titer and dividing by the fermentation time in hours. Yield was calculated by taking the oil titer and dividing by the mass of total glycerol (batch and feed) per liter.

### Results

The 40% feedstock outperformed the 30% feedstock which outperformed the defined media control in terms of DCW, oil titer, productivity, and yield. Lipid content, which is calculated by dividing the oil titer by the DCW, was comparable across all three feedstocks.

These results demonstrate that the feedstocks of the present disclosure can be used to produce high oil titers, over 30 g/L for the 40% feedstock condition, in the commercially relevant setting of fed batch fermentation.

### Example 5: Exemplary R. toruloides Growth on Different Feedstocks and Carbon Sources

### Growth on Different Post-Fermentation Feedstocks

Two exemplary R. toruloides strains, strain A and strain B, were fermented on three exemplary post-fermentation feedstocks of the present disclosure. The feedstocks were: 30% thin stillage—30% thin corn stillage diluted in deionized water; 100% whole stillage; and 30% clarified thin stillage—30% thin corn stillage diluted in deionized water and then clarified via centrifugation. The DCW results demonstrated that all three of these feedstocks were able to support cell growth for both strains of R. toruloides.

### Growth on Different Carbon Sources

Three exemplary strains of R. toruloides (strains A, B, and C) were grown on yeast peptone (YP) media (20 g/L peptone, 10 g/L yeast extract) with added arabinose, glucose, glycerol, sucrose, and xylose combined to determine the ability and preference of this species to consume different carbon sources. The carbon sources were added to equal initial concentrations of 12 g/L each, with a total carbon content of 60 g/L within the sample. The consumption of these carbon sources was measured via HPLC over time. The results of the analysis demonstrated that all three tested strains of R. toruloides could use any of the five carbon sources as fuel. All five carbon sources were consumed by R. toruloides strain A, with the general trend of preference in terms of consumption being: Glucose>Sucrose>Xylose/Fructose>Glycerol>Arabinose. These results indicate that three different exemplary strains of R. toruloides were able to utilize a variety of carbon sources as fuel for fermentation.

### Example 6: Comprehensive Analysis of an Illustrative Crude Microbial Oil Sample

A 100 g sample of crude microbial oil produced by the oleaginous microorganism R. toruloides was analyzed for general physical chemical characterization; fatty acid content, triglyceride content, diglyceride content, monoglyceride content, slip melting point, color; and contaminant (3-MCPD, GEs) levels. These analyses were carried out in comparison to standard Colombian palm oil and hybrid palm oil samples over the course of 70 days. Samples were stored in the dark, at cold temperatures, and at atmospheric nitrogen conditions.

The three oil samples were analyzed along different physical and chemical parameters, the results of which analyses are shown in Table 4. The methods employed were those of the American Oil Chemists' Society (AOCS) and are referenced within the Table by their AOCS identifier.

TABLE 4
General physical chemical characterization

| | | | | Crude | Crude | Crude |
|---|---|---|---|---|---|---|
| | | | | microbial | palm | hybrid |
| Parameter | Unit | Method | Equipment | oil | oil | palm oil |
| Free fatty acid | % | AOCS Ca 5a-40 | 865Dosimat plus | 2.58 | 2.81 | 2.02 |
| content | | | (Metrohm) | | | |
| Triglyceride | % | Arithmetical | — | 96.5 | 96.3 | 93.6 |
| content | | calculation | | | | |
| Diglyceride | % | AOCS Cd 11b-91 | GC-COC/FID | 0.94 | 5.49 | 4.04 |
| content | | | (7890A, Agilent) | | | |
| Monoglyceride | % | AOCS Cd 11b-91 | GC-COC/FID | <0.1 | <0.1 | <0.1 |
| content | | | (7890A, Agilent) | | | |
| Slip melting | ° C. | AOCS Cc 3-25 | Magnetic Stirrer | <15 | 36.2 | <15 |
| point | | | (MR-Hei-Std, | | | |
| | | | Heidolph) | | | |
| Color | red | AOCS Cc 13e-92 | Spectrocolorimeter | 46 | 28.4 | 39 |
| (Lovibond). Day 0. | | (cuvette 1″) | PFXi Series | | | |
| | yellow | | 995 (Lovibond) | 70 | 47 | 70 |

As shown in Table 4 above, crude microbial oil has similar amounts of free fatty acids, triglycerides, and monoglyceride as those found in crude palm oil and crude hybrid oil. Specific triglycerides were also measured and shown below.

Levels of contaminants were assessed in microbial oil, crude palm oil, and crude hybrid palm oil, with results shown in Table 5. The methods and equipment are shown in columns two and three, respectively.

TABLE 5
Contaminant levels

| | | | Crude | Crude | Crude |
|---|---|---|---|---|---|
| | | | microbial | palm | hybrid |
| Contaminant | Method | Equipment | oil | oil | palm oil |
| 3-MCPD | DGF C-VI | GC-SSL/MSD | <LOQ | <LOQ | <LOQ |
| | 18 (10) | (7890-5977A, | | | |
| | | Agilent) | | | |
| GEs | DGF C-VI | GC-SSL/MSD | <LOQ | <LOQ | <LOQ |
| | 18 (10) | (7890-5977A, | | | |
| | | Agilent) | | | |
| Phosphorus | AOCS Ca | Spectrophotometer | <1 ppm | 25 ppm | 20 ppm |
| content | 12-55 | UV-1280 | | | |
| | | (Shimadzu) | | | |

All three samples had contaminant levels below the limit of quantitation (LOQ). However, the samples differed greatly in the amount of phosphorous detected. Unlike crude palm oil and crude hybrid palm oil, which had 25 ppm and 20 ppm respectively, crude microbial oil had less than 1 ppm of phosphorous.

Whole microbial oil may be used in personal care items, for example as a replacement for any mineral oil or vegetable-derived oil. Additionally, it may be used as luxury soft oil in formulations of personal care compositions.

### Example 7: Comprehensive Analysis of Triglyceride Composition

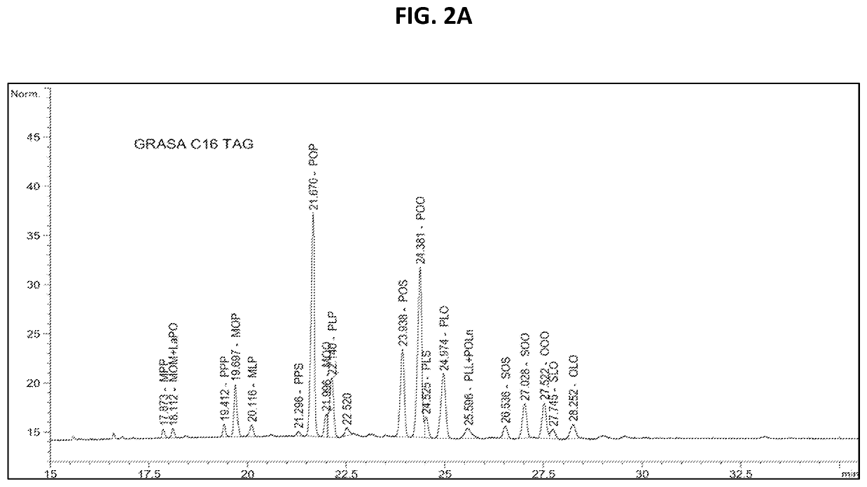

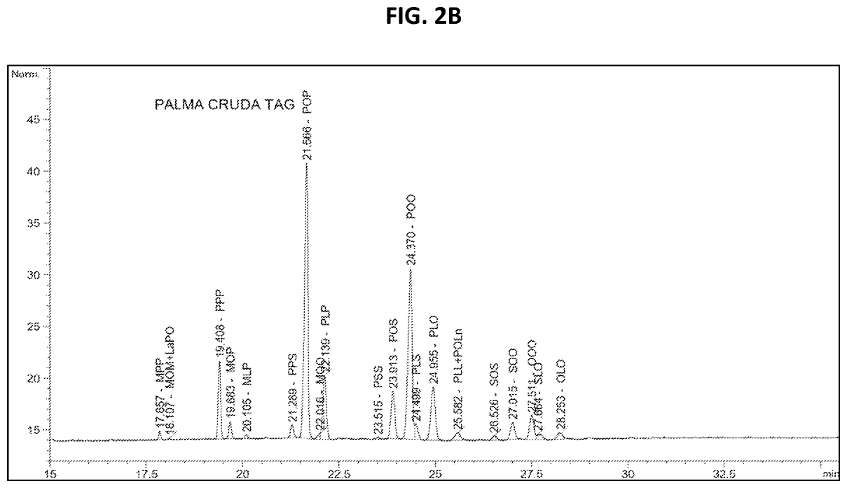

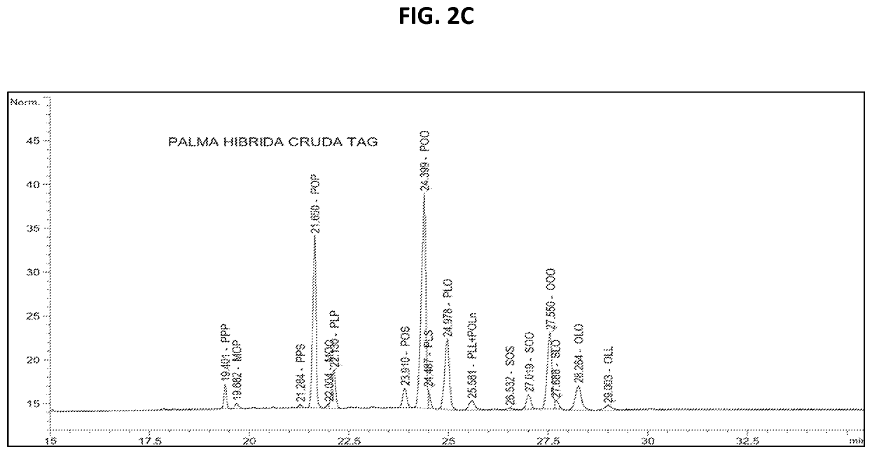

The triglyceride compositions of the three samples were analyzed on a GC-COC/FID (7890A, Agilent) instrument according to the AOCS Ce 5-86 method. Table 6 shows the results of the triglyceride analysis, with values as w/w percentages. The abbreviations used are as follows. M: Myristic fatty acid; S: Stearic fatty acid; P: Palmitic fatty acid; O: Oleic fatty acid; L: Linoleic fatty acid; La: Lauric fatty acid; Ln: linoleic fatty acid. The chromatogram for crude microbial oil is shown in FIG. 2A, the chromatogram for crude palm oil is shown in FIG. 2B, and the chromatogram for crude hybrid palm oil is shown in FIG. 2C.

TABLE 6
Triglyceride composition

| | | Crude | Crude | Crude |
|---|---|---|---|---|
| | | microbial | palm | hybrid |
| Triglyceride | Unit | oil | oil | palm oil |
| MPP | % | 0.65 | 0.60 | 0.00 |
| MOM + | % | 0.75 | 0.12 | 0.00 |
| LaPO | | | | |
| PPP | % | 1.02 | 6.48 | 2.11 |
| MOP | % | 4.73 | 1.58 | 0.55 |
| MLP | % | 1.27 | 0.35 | 0.00 |
| PPS | % | 0.43 | 1.38 | 0.35 |
| POP | % | 22.53 | 31.62 | 19.45 |
| MOO | % | 1.89 | 0.49 | 0.37 |
| PLP | % | 7.51 | 7.87 | 5.20 |
| PSS | % | 0.00 | 0.23 | 0.00 |
| POS | % | 10.25 | 6.11 | 2.68 |
| POO | % | 20.78 | 23.24 | 32.62 |
| PLS | % | 2.12 | 1.62 | 1.38 |
| PLO | % | 9.11 | 8.08 | 11.53 |
| PLL + | % | 2.04 | 1.41 | 1.78 |
| POLn | | | | |
| SSS | % | 0.00 | 0.00 | 0.00 |
| SOS | % | 1.53 | 0.60 | 0.29 |
| SOO | % | 4.29 | 2.46 | 2.29 |
| OOO | % | 4.54 | 3.63 | 12.17 |
| SLO | % | 1.30 | 0.98 | 1.09 |
| OLO | % | 2.33 | 1.14 | 4.93 |
| OLL | % | 0.00 | 0.00 | 1.23 |
| LLL | % | 0.00 | 0.00 | 0.00 |
| LLnL | % | 0.00 | 0.00 | 0.00 |
| LnLLn | % | 0.00 | 0.00 | 0.00 |
| LnLnLn | % | 0.00 | 0.00 | 0.00 |
| OOA | % | 0.00 | 0.00 | 0.00 |
| LLnLn | % | 0.00 | 0.00 | 0.00 |
| SOA | % | 0.00 | 0.00 | 0.00 |
| Total | % | 99.06219 | 100 | 100 |

The microbial oil sample showed similarity to both palm oil and hybrid palm oil along different parameters of fatty acid and triglyceride content. For example, microbial oil comprised approximately 1.2% w/w palmitic-palmitic-palmitic triglycerides, approximately 22.53% w/w palmitic-palmitic-oleic triglycerides, approximately 20.78% w/w oleic-oleic-palmitic triglycerides, approximately 1.53% w/w stearic-stearic-oleic triglycerides, and approximately 4.29% w/w stearic-oleic-oleic triglycerides.

### Example 8: Fractionated Microbial Olein and Stearin Production

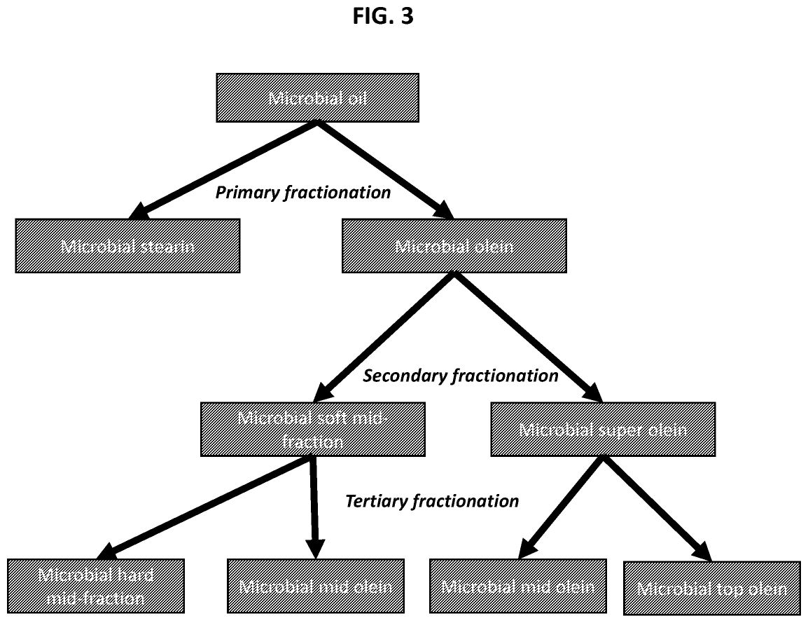

Fractionation of is another means of processing the microbial oil described herein for use in personal care compositions. Fractionation may be used to physically separate room temperature oil into saturated and unsaturated components. As shown in FIG. 3, the primary fraction of microbial oil results in microbial stearin and microbial olein. A secondary fraction of microbial olein results in microbial soft mid-fraction and microbial super olein. A tertiary fractionation of the soft mid-fraction results in a microbial hard mid-fraction and microbial mid-olein. A tertiary fractionation of the microbial super olein results in microbial mid-olein and microbial top olein.

The melting points of full oil mixtures and their saturated/unsaturated components differ. Hydrophilization makes use of surface active agents (surfactants) that dissolve solidified fatty crystals and emulsify liquid oils. By centrifuging this hydrophilized suspension, fats can be separated into different fractions based on saturation. Palm oil and microbial oil were fractionated and the saturation levels of their fractions were compared.

Crude palm oil and an R. toruloides microbial oil were fractionated using a method as set out in, e.g., Stein, W., “The Hydrophilization Process for the Separation of Fatty Materials,” Henkel and Cie, GmbH, Presented at AOCS Meeting, New Orleans, May 1967.

The oil sample was weighed and then incompletely melted to 50° C. The temperature was then brought down to 32° C. over the course of 10 min. The temperature was then slowly lowered to 20° C. with periods of time held at select temperatures between 32° C.-20° C. as follows: 32° C.-30 min; 26° C.-15 min; 24° C.-15 min; 22° C.-15 min; 21° C.-15 min; 20° C.-15 min. The oil sample was then maintained at 20° C. for an additional 1 hr.

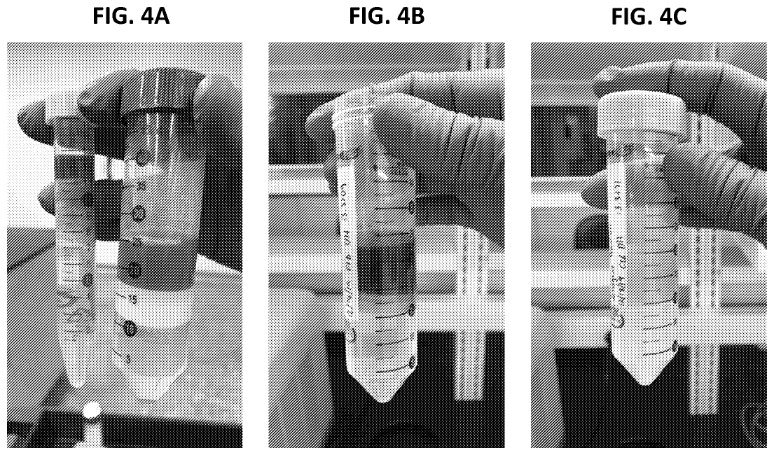

After this temperature manipulation, the oil sample was emulsified in a wetting agent solution at a ratio of 1:1.5 w/w fat to wetting agent. The wetting agent was comprised of a salt and a detergent in DI water: 0.3% (w/w) sodium lauryl sulfate; 4% (w/w) magnesium sulfate. The oil/wetting agent mixtures were vortexed until thoroughly mixed. The samples were centrifuged at 4700 rpm for 5 min in a benchtop centrifuge. The lighter oil phase migrated to the top, while the heavier aqueous phase (containing solid, saturated fatty particles) migrated to the bottom. Shown in FIG. 4A is a photograph of a fractionation of crude microbial oil (left) and crude palm oil (right). The top olein layer is liquid, and the bottom stearin layer is solid. FIG. 4B is a photograph of a complete fractionation of crude microbial oil, and FIG. 4C is a photograph of an incomplete fractionation of crude microbial oil.

The aqueous phase was separated by aspirating the upper olein phase into a pre-weighed scintillation vial. The aqueous phase was heated—with its solidified stearin layer interspersed atop—until all fatty materials melted. This heated aqueous phase was centrifuged (4700 rpm, 1 min, 40° C.) and the stearin fraction was also aspirated into a pre-weighed scintillation vial.

The separated olein and stearin fractions were weighed and their masses compared to the original mass of oil pre-fractionation. By mass, an exemplary microbial oil produced by R. toruloides was 68.4% w/w olein and 31.6% w/w stearin. By comparison, a crude plant-derived palm oil sample was analyzed as comprising 72% w/w olein and 28% w/w stearin using this fractionation method.

Next, the iodine value (IV) for each fraction was calculated, which is expressed as the number of grams of iodine absorbed by 100 g of the oil sample. The microbial olein fraction had an iodine value of 81 and the microbial stearin fraction had an iodine value of 22. The crude palm oil olein fraction had an IV of 53 and the stearin fraction had an IV of 40. These results indicate an even more distinct fractionation of saturated and unsaturated fatty acids between the microbial fractions, a distinction that could be useful for the manufacture of downstream products, as plant-derived palm oil may require multiple fractionation steps to achieve this level of differentiation between fractions.

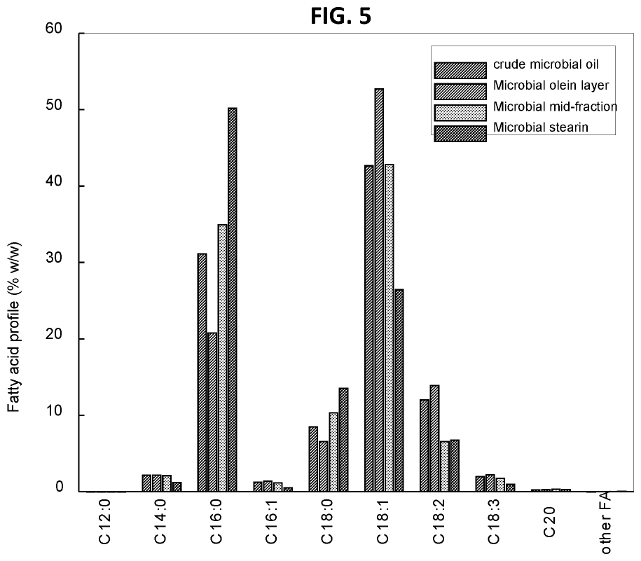

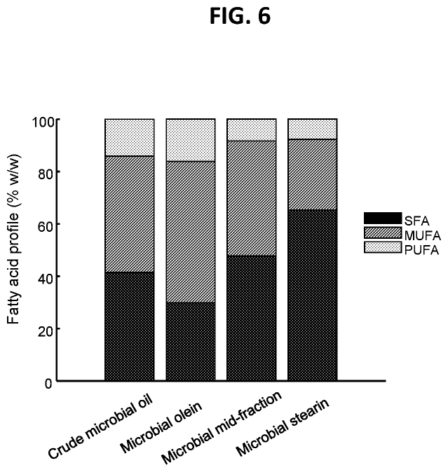

The fatty acid profile of the fractioned oil was also analyzed. Shown in FIG. 5 is a bar graph of the fatty acid profile of crude microbial oil, microbial olein layer, microbial mid-fraction, and microbial stearin layer. Shown in FIG. 6 is a bar graph of the saturated profiles of crude microbial oil, microbial olein layer, microbial mid-fraction, and microbial stearin layer. As shown in FIG. 6, Microbial olein has a greater percentage of monounsaturated fatty acids compared to microbial stearin, which has a greater percentage of saturated fatty acids. The microbial stearin fractions shown are solid at room temperature (slip melting point >25 ° C.), whereas the olein fractions are liquid at room temperature. In some instances, microbial fractionation gives rise to three layers: a stearin, olein, and mid-fraction. In some instances, the microbial oil may be re-fractioned to generate double stearin, or double olein, for example. Thus, the microbial oil of the present disclosure may be fractioned similar to other plant-derived oils, such as palm oil.

Additionally, fractionated microbial olein and fractionated microbial stearin may replace rice bran oil and shea butter, respectively in personal care compositions. Fractioned olein may also replace vegetable oils high in oleic acids, such as tea seed oil.

### Example 9: Ester Production

Esterification is the general name for a reaction that generates esters, a compound derived from an acid. In some embodiments, the disclosure relates to esters derived from fatty acids produced by oleaginous yeast, wherein the esters are used in a personal care composition.

Oil samples were converted into fatty acid methyl esters (FAMEs) and then analyzed using gas chromatography-mass spectrometry (GC-MS). A method of using commercial aqueous concentrated HCl (conc. HCl; 35%, w/w) as an acid catalyst was employed for preparation of fatty acid methyl esters (FAMEs) from microbial oil and palm oil for GC-MS. FAME preparation was conducted according to the following exemplary protocol.

Commercial concentrated HCl (35%, w/w; 9.7 ml) was diluted with 41.5 ml of methanol to make 50 ml of 8.0% (w/v) HCl. This HCl reagent contained 85% (v/v) methanol and 15% (v/v) water that was derived from conc. HCl and was stored in a refrigerator.

A lipid sample was placed in a screw-capped glass test tube (16.5×105 mm) and dissolved in 0.20 ml of toluene. To the lipid solution, 1.50 ml of methanol and 0.30 ml of the 8.0% HCl solution were added in this order. The final HCl concentration was 1.2% (w/v) or 0.39 M, which corresponded to 0.06 ml of concentrated HCl in a total volume of 2 ml. The tube was vortexed and then incubated at 45° C. overnight (14 h or longer) for mild methanolysis/methylation or heated at 100° C. for 1 h for rapid reaction. After cooling to room temperature, 1 ml of hexane and 1 ml of water were added for extraction of FAMEs. The tube was vortexed, and then the hexane layer was analyzed by GC-MS directly or after purification through a silica gel column.

For the analysis of fatty acid composition, a Shimadzu GCMS-TQ8040/GC-2010 Plus instrument was employed. The FAME samples were concentrated at 5 g/L in hexane/chloroform/heptane prior to analysis.

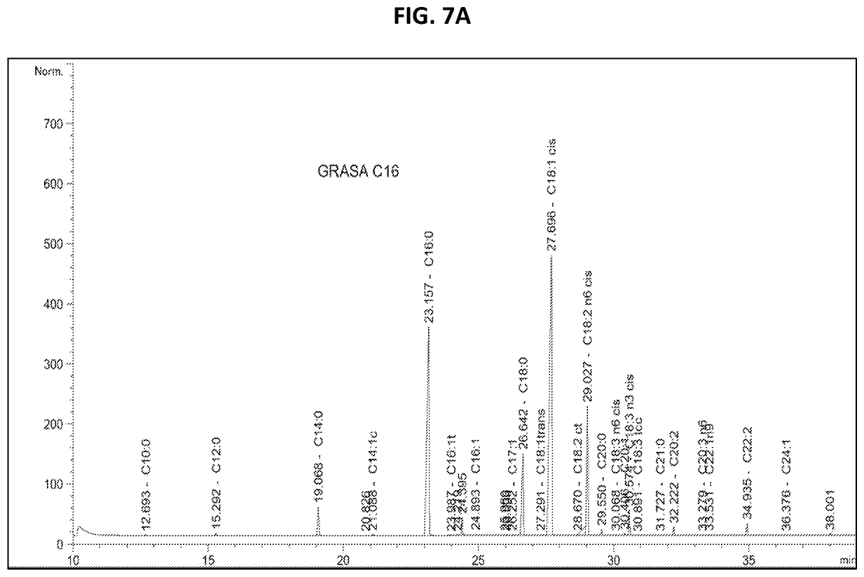

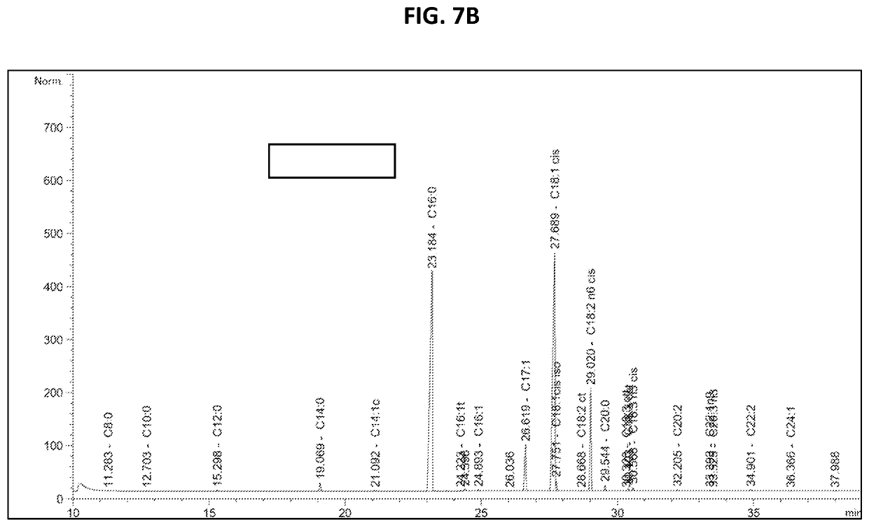

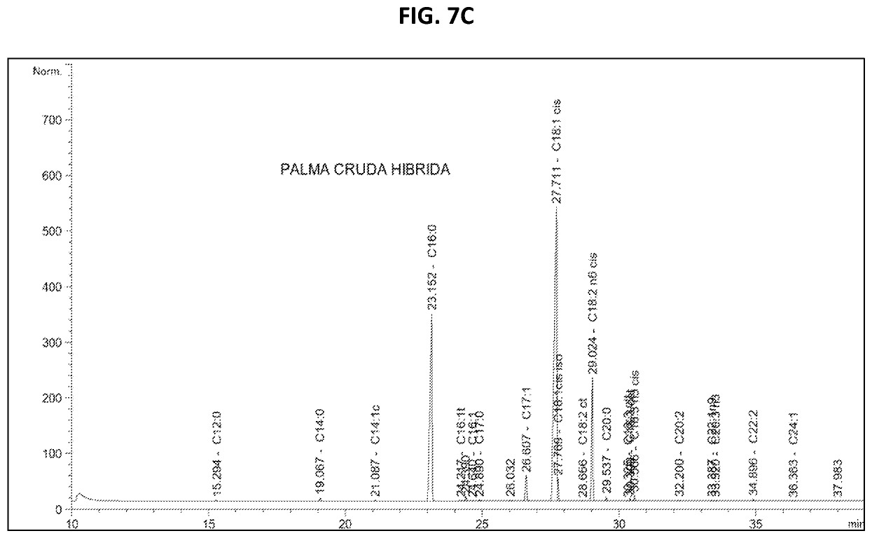

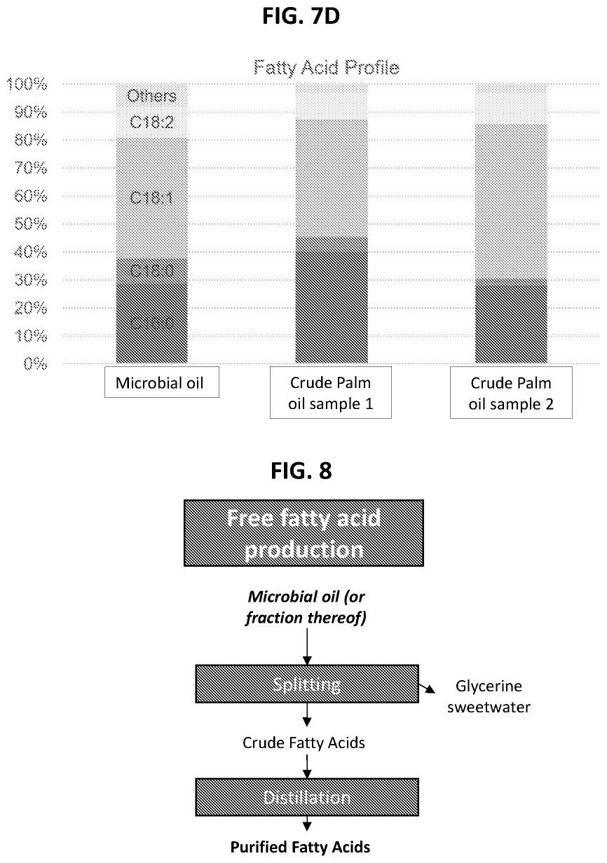

The results of the analysis are shown in Table 7 comparing the fatty acid composition of three exemplary microbial oil samples produced by Rhodosporidium toruloides to the measurements expected for crude palm oil, as set forth by guidelines from the Malaysian government. For Microbial oil sample 3, the fatty acid compositions were determined via fatty acid methyl ester analysis with a GC-SSL/FID (7890A, Agilent) instrument. The methods employed were using AOCS Ce la-13 and AOCS C2 2-66. (see also FIG. 7A-7D). Table 7 shows the breakdown of the individual fatty acid constituents by w/w percent, with the percentages for each sample adding up to 100%. Fatty acids that were assayed but not detected in any sample include C4, C6, C13, C15, C15:1, C18:2 tt, C18:2 5,9, C18:2 tc, C18:3, C18:3 ctc, C18:3 ttt, C18:3 ttc+tct, C20:4 n6ARA, C22, and C24.

TABLE 7
Fatty acid composition of microbial oil samples

| Fatty | Microbial oil | Microbial oil | Microbial oil | Palm oil | Palm oil |
|---|---|---|---|---|---|
| Acid | Sample 1 | Sample 2 | sample 3 | MIN | MAX |
| C8:0 | 0.0% | 0.0% | 0.0% | 0.0% | 0.1% |
| C10:0 | 0.0% | 0.0% | 0.04% | 0.0% | 0.1% |
| C12:0 | 0.2% | 0.0% | 0.17% | 0.0% | 0.5% |
| C14:0 | 1.8% | 1.7% | 2.24% | 0.5% | 2.0% |
| C15:1 | 0.5% | 0.5% | 0.0% | 0.0% | 0.1% |
| C16:0 | 14.5% | 13.8% | 28.7% | 39.3% | 47.5% |
| C16:1 | 0.6% | 0.7% | 0.10% | 0.0% | 0.6% |
| C17:0 | 10.2% | 9.5% | 0.0% | 0.0% | 0.2% |
| C17:1 | 0.8% | 0.6% | 0.03% | 0.0% | 0.1% |
| C18:0 | 26.9% | 28.8% | 8.98% | 3.5% | 6.0% |
| C18:1 | 10.0% | 16.3% | 43.39% | 36.0% | 44.0% |
| C18:2 | 15.2% | 16.1% | 10.77% | 9.0% | 12.0% |
| C20:0 | 8.3% | 3.6% | 0.0% | 0.0% | 0.0% |
| C18:3 | 0.2% | 0.0% | 1.75% | 0.0% | 0.5% |
| C20:1 | 2.5% | 0.4% | 0.13% | 0.0% | 0.4% |
| C22:0 | 2.6% | 0.7% | 0.0% | 0.0% | 0.2% |
| C22:1 | 0.3% | 0.3% | 0.02% | 0.0% | 0.0% |
| C22:2 | 0.3% | 0.0% | 0.94% | 0.0% | 0.0% |
| C24:0 | 5.0% | 7.1% | 0.0% | 0.0% | 0.0% |
| Other | | | 2.74% | | |

These results show that exemplary microbial oil samples of the present disclosure have a similar breakdown of saturated vs. unsaturated fatty acids compared to plant-derived palm oil, though the specific identities of the predominant fatty acids differs between the microbial samples and typical palm oil. Similar to palm oil, though, C16:0 was a significant source of saturated fatty acid in the microbial samples and C18 unsaturated fatty acids made up the majority of the unsaturated fatty acids in the sample.

The fatty acid composition breakdown of the samples were determined via fatty acid methyl ester analysis with a GC-SSL/FID (7890A, Agilent) instrument. The methods employed were using AOCS Ce la-13 and AOCS C2 2-66. The results these analyses are shown in Table 8. Table 8 below shows the breakdown of the individual fatty acid constituents by w/w percent, with the percentages for each sample adding up to 100%. Fatty acids that were assayed but not detected in any sample include C4, C6, C13, C15, C15:1, C18:2 tt, C18:2 5,9, C18:2 tc, C18:3, C18:3 ctc, C18:3 ttt, C18:3 ttc+tct, C20:4 n6ARA, C22, and C24.

TABLE 8
Fatty acid composition breakdown

| Condensed | Common | Crude microbial | Crude palm | Crude hybrid |
|---|---|---|---|---|
| formula | name | oil | oil | palm oil |
| C8:0 | Caprylic | | 0.01% | |
| C10:0 | Capric | 0.04% | 0.01% | |
| C11:0 | Undecylic | 0.00% | | |
| C12:0 | Lauric | 0.17% | 0.11% | 0.08% |
| C14:0 | Myristic | 2.24% | 0.75% | 0.27% |
| C14:1c | Myristoleic | 0.08% | 0.05% | 0.06% |
| C16:0 | Palmitic | 28.70% | 40.20% | 27.79% |
| C16:1t | | 0.01% | 0.04% | 0.05% |
| C16:1 | Palmitoleic | 0.10% | 0.10% | 0.01% |
| C17:0 | Margaric | | | 0.08% |
| C17:1 | | 0.03% | | |
| C18:0 | Stearic | 8.98% | 5.15% | 2.65% |
| C18:1trans | | 0.08% | | |
| C18:1 cis | Oleic | 43.39% | 42.09% | 55.21% |
| C18:1cis iso | | | 0.59% | 1.11% |
| C18:2 ct | | 0.07% | 0.01% | 0.01% |
| C18:2 n6 cis | Linoleic | 10.77% | 9.61% | 11.23% |
| C18:3 ctt | | | 0.01% | 0.01% |
| C20:0 | Arachidic | 0.35% | 0.41% | 0.28% |
| C18:3 cct | | | 0.15% | 0.15% |
| C18:3 n6 cis | γ-Linolenic | 0.05% | | |
| | (GLA) | | | |
| C18:3 tcc | | 0.01% | | |
| C20:1 | | 0.13% | | |
| C18:3 n3 cis | α-Linolenic | 1.69% | 0.30% | 0.39% |
| | (ALA) | | | |
| C21:0 | heneicosylic | 0.03% | | |
| C20:2 | cis-11,14- | 0.54% | 0.07% | 0.06% |
| | eicosadienoic | | | |
| C20:3 n6 | | 0.02% | | |
| C22:1n9 | Erucic | 0.02% | 0.05% | 0.04% |
| C20:3 n3 | | | 0.02% | 0.02% |
| C22:2 | | 0.94% | 0.09% | 0.10% |
| C24:1 | | 0.04% | 0.01% | 0.01% |
| Unknown | | 1.20% | | |

Table 9 shows the w/w percentage of saturate, trans, mono-unsaturated, poly-unsaturated, and unknown fatty acids in each sample. The fatty acid compositions were determined via fatty acid methyl ester analysis with a GC-SSL/FID (7890A, Agilent) instrument. The methods employed were using AOCS Ce 1a-13 and AOCS C2 2-66. FIG. 7A-7C show the chromatograms for the crude microbial oil (FIG. 7A), palm oil (FIG. 7B), and hybrid palm oil (FIG. 7C), respectively. FIG. 7D shows a bar graph of representative compositions of microbial oil and palm oil.

TABLE 9
Overall fatty acid composition

| | Crude | Crude | Crude hybrid |
|---|---|---|---|
| | microbial oil | palm oil | palm oil |
| Saturated fatty acid | 40.5% | 41.5% | 28.5% |
| Trans fatty acid | 0.17% | 0.21% | 0.22% |
| Mono-unsaturated | 43.8% | 48% | 59.1% |
| fatty acid | | | |
| Poly-unsaturated | 14% | 10.1% | 11.8% |
| fatty acid | | | |
| Unknown | 1.5% | 0% | 0% |

As shown in the above and described herein, oleaginous microbial oil has a similar profile to that of palm oil, and thus esters derived from palm oil and used in personal care items may be substituted for esters derived from oleaginous yeast. Methods of producing esters from fatty acids are well known in the art. See, for example, Milinsk, M. C. et al., Comparative analysis of eight esterification methods in the quantitative determination of vegetable oil fatty acid methyl esters (FAME), J. Braz. Chem. Soc., 2008, vol.19, n.8. One example use of the microbial oils described herein is esterification of a specific fatty acid produced from an oleaginous yeast, and use of the resultant ester as an ingredient in a personal care composition.

As shown in Table 8, oleaginous yeast produced approximately 9% stearic acid. Esterification of stearic acid produced by the oleaginous yeast described herein can produce stearate esters for use as emollients and thickeners in personal care items. For example, a reaction with ethylhexyl alcohol can produce Ethylhexyl Stearate (also known as Octyl Stearate). Similarly, reactions with other alcohols can produce Butyl Stearate, Cetyl Stearate, Isocetyl Stearate, Isopropyl Stearate, Myristyl Stearate, and Isobutyl Stearate, and Octyldodecyl Stearoyl Stearate.

Stearate esters may be used in personal care compositions such as, for example, cosmetics, such as eye makeup, skin makeup, and lipstick, skin care products, such as lotions and sunscreens, hair conditioners, hair styling products, and nail polish.

As shown above in Table 8 above, oleaginous yeast produced approximately 28.7% palmitic acid. Esterification of palmitic acid produced by the oleaginous yeast described herein can produce palmitate esters. For example, a reaction with ethylhexyl alcohol can produce ethylhexyl palmitate (also known as Octyl Palmitate), which may act as an emollient in a composition. Similarly, Isopropyl Palmitate is the ester of isopropyl alcohol and palmitic acid, and can function as an emollient, emulsifier, stabilizer (for example, in antiperspirant sticks), film former, spreader, and a solvent in creams, lotions, and eye makeup. It may also be found as an ingredient in aftershave products. Cetyl Palmitate is the ester of cetyl alcohol and palmitic acid, and can function as an emollient in a composition. For example, Cetyl Palmitate can contribute to the texture of a cream or lotion, serve as a base for ointments, and may be an ingredient in soaps. Isostearyl Palmitate is the ester of isostearyl alcohol and palmitic acid, and can work as an emollient and help eliminate the greasy feel of oils and heavy esters. For additional examples of Palmitates used in personal care products and ranges of their concentrations in various products see Final Report on the Safety Assessment of Octyl Palmitate, Cetyl Palmitate and Isopropyl Palmitate, Journal of the American College of Toxicology, 1990: 1(2): 13-35.

### Example 10: Free Fatty Acid and Fatty Alcohol Production

Hydrolysis is the process whereby triglycerides in fats and oils are split (“fat splitting” or “oil splitting”) into glycerol and fatty acids. It is usually carried out using great amounts of high-pressure steam (“steam hydrolysis”) but may also be performed using catalysts (for example, the tungstated zirconia and solid acid composite SAC-13 (Hydrolysis of Triglycerides Using Solid Acid Catalysts, Ngaosuwan, K, et al., Ind. Eng. Chem. Res., 2009 48 (10), 4757-4767)). The reaction proceeds in a step-wise fashion wherein fatty acids on triglycerides are displaced one at time, generating diglycerides, then monoglycerides, and finally free fatty acids and glycerin.

Shown in FIG. 8 is a flow diagram of an example method to produce purified fatty acids from microbial oil or fractions thereof. The crude microbial oil may first be deaerated to remove un-dissolved gasses. Next, the fatty acids are produced by steam hydrolysis, wherein the temperature is raised up to 260 degrees Celsius at a pressure of 60 bar. Glycerine may be collected and further purified for various uses, and the crude fatty acids are subsequently purified by distillation. Fatty acids may be further modified to produce, for example, conjugated fatty acids, dimer acids, fatty acids ethoxylates, and fatty acid esters.

Examples of fatty acids derived from oleaginous microorganisms that may be used in personal care composition include, but are not limited to, stearic acid, oleic acid, palmitic acid, and myristic acid.

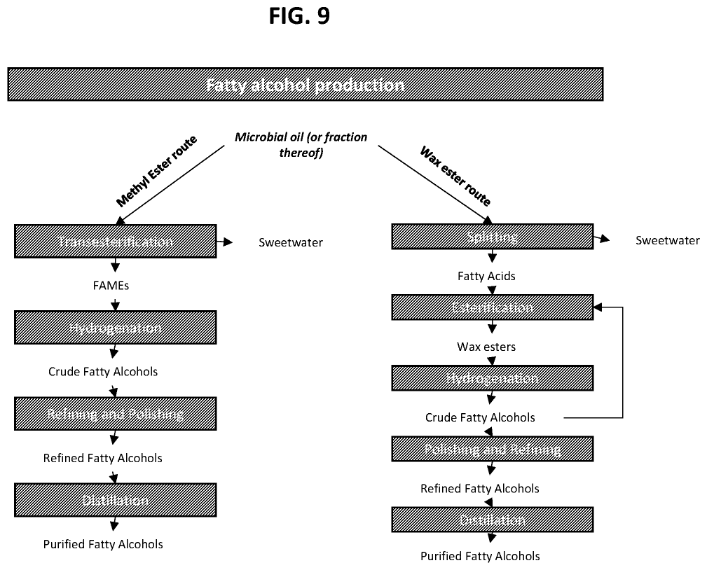

Fatty alcohols may be produced via a methyl ester route or a wax ester route (FIG. 9). In the methyl ester route (also known as the Davy process), FAMEs produced by transesterification may be hydrogenated to produce crude fatty alcohols, which are then refined, polished, and purified. In the wax ester route, (also known as the Lurgi process) fatty acids produced from hydrolysis (“splitting”) are further modified via esterification to produce wax esters, which may then be hydrogenated to produce fatty alcohols. Direct hydrogenation of fatty acids is also possible and produces fatty alcohols. Fatty alcohols may be further modified to produce, for example, fatty alcohol ethoxylates, and fatty alcohol sulfates.

Examples of fatty alcohols derived from oleaginous microorganisms that may be used in personal care compositions include, but are not limited to, cetearyl alcohol, cetyl alcohol, isostearyl alcohol, and myristyl alcohol.

### Example 11: Fatty Acid Salt Production

Saponification is the process whereby triglycerides or free fatty acids used as feedstock are converted to fatty acids salts (soaps), glycerol, and free fatty acids in the presence of a base. The base may be for example, sodium hydroxide, or potassium hydroxide. Saponification may be achieved via a hot or cold process. The cold process uses the heat generated from the combination of the fatty acids in the melted oils and fats with sodium hydroxide (base). This process takes longer, and an additional curing phase is needed for the soap to harder. The hot process uses heat to speed up the saponification process, and generally no additional curing step is required before use of the soap. Methods of saponification are well known in the art. See for example, Prieto Vidal N, et al., The Effects of Cold Saponification on the Unsaponified Fatty Acid Composition and Sensory Perception of Commercial Natural Herbal Soaps. Molecules. 2018;23(9):2356.

The triglycerides or free fatty acids described herein (see for example, Table 8) may be used in a saponification reaction to produce salts, glycerin, and free fatty acids. For example, sodium stearate is produced by saponification of stearic acid, and it is one of the most commonly used commercial surfactants in soap. Sodium oleate is produced by the saponification of oleic acid. Saponification of palmitic acid produces sodium palmitate. Potassium stearate is the potassium salt of stearic acid. Metal salts may also be produced, for example, zinc stearate and magnesium myristate.

As will be understood by one skilled in the art, saponification of the triglycerides disclosed herein may produce a number of salts and glycerin for use in personal care compositions.

### Unsaponifiable Lipid Content

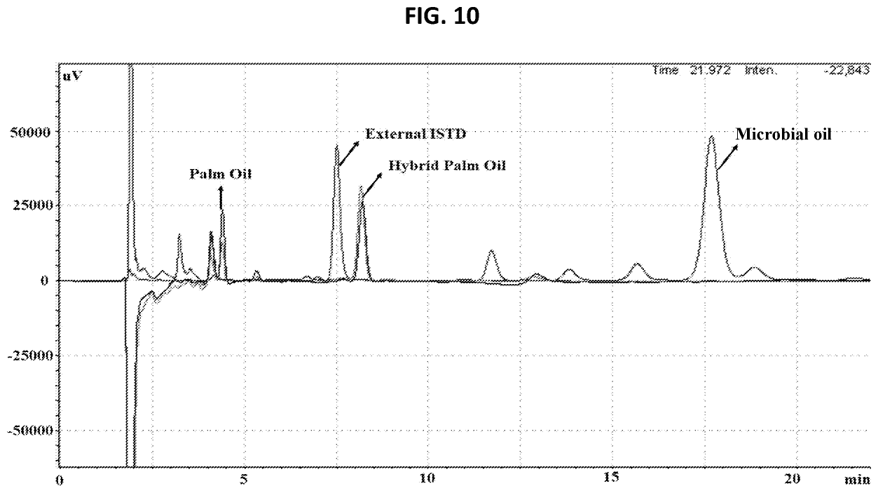

The unsaponifiable lipid content of the three microbial oil samples was analyzed, specifically measuring the amount of beta-carotene (data not shown), squalene, tocopherols, and sterols in each sample. Results are shown in Table 10. Beta-carotene was analyzed using the method of Luterotti et al., “New simple spectrophotometric assay of total carotenes in margarines,” Analytica Chimica Acta 2006; 573:466-473, incorporated by reference herein in its entirety. The sterol composition was analyzed using the method of Johnsson et al., “Side-chain autoxidation of stigmasterol and analysis of a mixture of phytosterol oxidation products by chromatographic and spectroscopic methods,” Journal of the American Oil Chemists' Society 2003; 80(8):777-83, incorporated by reference herein in its entirety, with the HPLC-DAD chromatogram results shown in FIG. 10. The other methods that were employed are indicated in Table 10. The sterol composition of the microbial oil sample showed an atypical sterols chromatographic profile differentiating it from the palm oil and hybrid palm oil samples and warranting further investigation. In this illustrative sample, the unexpected sterol composition acts as a unique fingerprint for the microbial oil sample.

TABLE 10
Unsaponifiable lipid content

| | | | Crude | Crude | Crude |
|---|---|---|---|---|---|
| | | | microbial | palm | hybrid |
| Parameter | Method | Equipment | oil | oil | palm oil |
| Squalene | AOCS Ce | GC-SSL/FID | 122 | 389 | 260 |
| (ppm) | 1a-13 | (7890A, | | | |
| | | Agilent) | | | |
| Tocopherols | AOCS | LC-DAD/RID | <10 | 869 | 761 |
| (ppm) | Ce 8-89 | (Prominence, | | | |
| | | Shimadzu) | | | |
| Sterols (%) | Johnsson | GC-COC/FID | Unexpected | 0.07 | 0.1 |
| | et al. | (7890A, | profile | | |
| | | Agilent) | | | |

As shown in Table 10, the microbial oil sample does not contain significant levels of unsaponifiable lipids, or tocopherols. Specifically, microbial oil has approximately 122 ppm of squalene, compared to 389 ppm and 260 ppm in palm oil and hybrid palm oil respectively. Microbial oil also contained less than 10 ppm of tocopherols, whereas palm oil and hybrid palm oil contained 869 ppm and 761 ppm respectively.

### Example 12: Microbial Oil Used to Produce Personal Care Products Described in Examples 13-15

Microbial oil was prepared using R. toruloides fermented on glycerol feed, lysed with acid, and extracted with heptane solvent. The composition of the oil is shown below in Table 11.

TABLE 11
Oil batch analysis

| | Sample value |
|---|---|
| Odor | fatty odor |
| Description | red liquid at or above 17° C. |
| Acid Value (% Oleic) | 2.41 |
| Color (Yellow/Red, 1″) | 50.0/20.0 |
| Refractive Index | 1.464 |
| Iodine Value (GI/100 g) | 69.1 |
| Peroxide Value (mEq/kg) | 0.91 |
| Density (g/cc) | 0.904 |
| Saponification Value (mg KOH/g) | 194 |
| Melting Point (° C.) | 21 |
| C10:0 Capric (%) | 0.23 |
| C12:0 Lauric (%) | 0.00 |
| C14:0 Myristic (%) | 1.27 |
| C16:0 Palmitic (%) | 28.10 |
| C16:1 Palmitoleic acid (%) | 1.10 |
| C18:0 Stearic (%) | 8.19 |
| C18:1 Oleic (%) | 47.33 |
| C18:2 Linoleic (%) | 10.68 |
| C18:3 Linolenic (%) | 1.60 |
| C20:0 Arachidic (%) | 1.50 |
| SFA (%) | 39.29 |
| MUFA (%) | 48.43 |
| PUFA (%) | 12.28 |

### Example 13: Cleansing Oil Made With Microbial Oil and a Derivative Thereof

Oil cleansers are increasing in popularity with their ability to effectively break down make-up and dirt without any of the harsh surfactants that tend to strip skin of its natural moisture and cause dryness and irritation. Two versions of an oil cleanser were made using the recipe below in Table 12. Both comprised whole microbial oil isolated and prepared as described above in Example 12, but version 1 also had a derivative (isostearyl palmitate) isolated from microbial oil, while version 2 comprised isostearyl palmitate from palm. All values are shown as % w/w.

TABLE 12
Cleansing oil made with microbial oil and a derivative thereof

| | Version 1 | Version 2 |
|---|---|---|
| Caprylic Capric Triglyceride (CCT oil) | 59.6 | 59.6 |
| Isostearyl Palmitate (derived from the | 10.0 | |
| microbial oil described herein) | | |
| Isostearyl Palmitate (derived from palm) | | 10.0 |
| Salacos ® PG-218 | 14.0 | 14.0 |
| Salacos ® DG-158 | 6.0 | 6.0 |
| Covi-ox ® T50 | 0.1 | 0.1 |
| SymLite ® G8- Glyceryl Caprylate | 0.3 | 0.3 |
| Microbial oil (with 0.1% Covi-ox ® T50) | 10.0 | 10.0 |

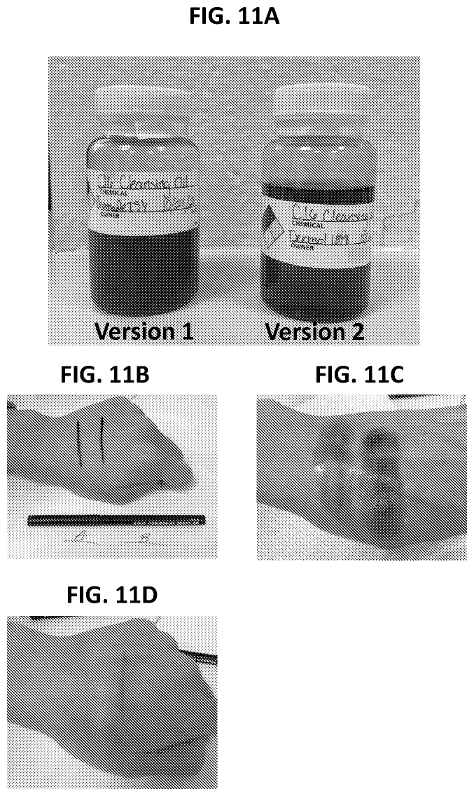

The resulting cleansing oils are shown in FIG. 11A. To test the efficacy of each cleanser, two lines (approximately 2 ½ ″ in length) of eye liner (e.l.f. No Budge Retractable Liner in black) were drawn across the top of a hand, labelled A & B (FIG. 11B). Two drops of the cleansing oil—one drop on the top of the line and one drop on the bottom of the line, were applied using version 1 for line A (comprising the isostearyl palmitate from microbial oil) and version 2 (comprising the palm based isostearyl palmitate) for line B. The cleansing oil on each line of eye liner was then rubbed (equally) (FIG. 11C) and then wiped with a dry paper towel (FIG. 11D). Similar experiments were performed with mascara and eye shadow (data not shown), and in approximately 50% of the cases, the formulation made with the biobased isostearyl palmitate showed a slight improvement in make-up removal vs the palm based sample.

### Example 14: Repair Serum Made With Microbial Oil and a Derivative Thereof

A skin repair serum was made using the microbial oil as described herein (prepared as described above in Example 12) and a derivative thereof, which combined the microbial oil with a number of other skin beneficial ingredients. Two versions of the repair serum were made using the recipe below in Table 13. Version 1 comprised a derivative (isostearyl palmitate) isolated from microbial oil, while version 2 comprised isostearyl palmitate from palm. All values are shown as % w/w.

TABLE 13
Skin repair serum made with microbial oil and a derivative thereof

| | Version 1 | Version 2 |
|---|---|---|
| Microbial oil (with 0.1% Covi-ox ® T50) | 10.0 | 10.0 |
| Jojoba oil | 5.0 | 5.0 |
| Isostearyl Palmitate (derived from the | 20.0 | |
| microbial oil described herein) | | |
| Isostearyl Palmitate (derived from palm) | | 20.0 |
| Caprylic Capric Triglyceride (CCT oil) | 63.9 | 63.9 |
| Covi-ox ® T50 | 0.1 | 0.1 |
| SabiWhite ® | 0.5 | 0.5 |
| Sytenol ® A-Bakuchiol | 0.5 | 0.5 |

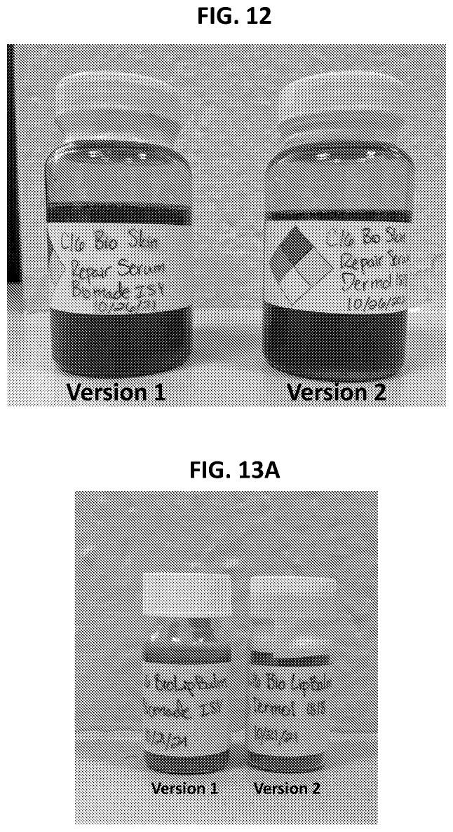

The resulting repair serums are shown in FIG. 12. The two serums performed similarly in terms of spreadability, slip, sheen/glow and skin coloration, however about 50% of the time version 1 (comprising the the isostearyl palmitate from microbial oil) was found to have a better feel (smooth, silky), whereas version 2 (comprising the palm based isostearyl palmitate) was found to have a more tacky feel.

### Example 15: Lip Balm Made With Microbial Oil and a Derivative Thereof

Lip balm was made using the microbial oil as described herein (prepared as described above in Example 12). Two versions of the lip balm were made using the recipe below in Table 14. Version 1 also comprised a derivative (isostearyl palmitate) isolated from microbial oil, while version 2 comprised isostearyl palmitate from palm. All values are shown as % w/w.

TABLE 14
Lip balm made with microbial oil and a derivative thereof

| | Version 1 | Version 2 |
|---|---|---|
| Rice bran wax | 15.0 | 15.0 |
| Cocoa butter | 10.0 | 10.0 |
| Coconut oil | 54.9 | 54.9 |
| Microbial oil (with 0.1% Covi-ox ® T50) | 10.0 | 10.0 |
| Isostearyl Palmitate (derived from the | 10.0 | |
| microbial oil described herein) | | |
| Isostearyl Palmitate (derived from palm) | | 10.0 |
| Covi-ox ® T50 | 0.1 | 0.1 |

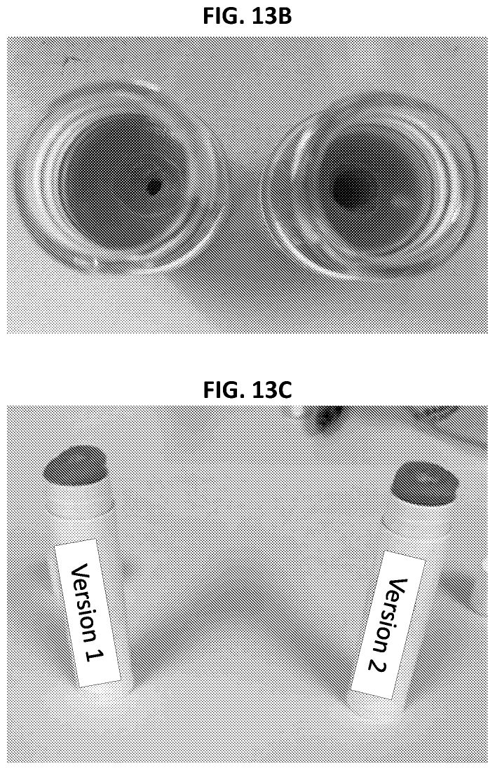

The resulting lip balms are shown in FIGS. 13A-13C. The lip balms were evaluated for stick firmness, payoff on skin, feel, slip, gloss/glow, sheen, and color of the skin. The products performed similarly in terms of stick firmness, payoff, feel, slip, gloss, glow/sheen, and skin coloration. However about 50% of the time version 1 comprising the microbial derivative rubbed in more easily, and about 50% of the time version 2 comprising the palm based derivative had more slip and delivered more color to the skin.

Based on the results of the evaluations that were done on the three products described above (Examples 13-15), the microbial based isostearyl palmitate performed similarly to the palm based benchmark, which shows that the microbial derivatives can be used as a replacement for palm based derivatives in personal care products.

### Example 16: Additional Products That May be Produced Using Microbial Oil and/or Derivatives

Listed below are several example recipes for various personal care items using the microbial oil and/or derivatives thereof described herein as ingredients, which may comprise anywhere between 0.1% to 100% of the composition. The recipes below are examples and should not be construed as limiting. Rather, these examples are provided so that this disclosure will be thorough and complete. Additional uses for the microbial oils and derivatives thereof, and various modifications to the example recipes below will be readily apparent to those skilled in the art. Specifically, any composition comprising a vegetable oil, (for example, palm oil, palm kernel oil, coconut oil, cocoa butter, shea butter, etc.) or derivatives thereof, may be substituted for a microbial oil or derivative thereof.

### Liquid Hand Soap

Water
Sodium laureth sulfate
Cocamidopropyl betaine
Sodium chloride
Glycol stearate (derived from an oleaginous microorganism)
Glycerin (derived from an oleaginous microorganism)
Sodium lauryl sulfate
Sodium lauroyl lactylate
Fragrance

### Bar Soap

Sodium lauroyl isethionate
Stearic acid (derivedfrom an oleaginous microorganism)
Lauric acid (derivedfrom an oleaginous microorganism)
Sodium oleate (derived from an oleaginous microorganism)
Water
Sodium isethionate
Sodium stearate (derivedfrom an oleaginous microorganism)
Cocamidopropyl betaine
Sodium chloride
Fragrance

### Hand Lotion

Water
Glycerin (derived from an oleaginous microorganism)
Stearic acid (derivedfrom an oleaginous microorganism)
Cetyl alcohol (derivedfrom an oleaginous microorganism)
Potassium stearate (derived from an oleaginous microorganism)
Shea butter or microbial butter (derived from an oleaginous microorganism)
Isopropyl palmitate (derivedfrom an oleaginous microorganism)
Whole microbial oil (derived from an oleaginous microorganism)
Orange peel oil
Potassium sorbate

### Facial Lotion

Water
Glycerin (derived from an oleaginous microorganism)
Stearic Acid (derivedfrom an oleaginous microorganism)
Emulsifying Wax (derivedfrom an oleaginous microorganism)
Fractionated microbial olein (derivedfrom an oleaginous microorganism)
Fractionated microbial stearin (derivedfrom an oleaginous microorganism)
Tea tree oil
Preservative

### Facial Balm

Fractionated microbial stearin (derivedfrom an oleaginous microorganism)
Fractionated microbial olein (derivedfrom an oleaginous microorganism)
Beeswax
Jojoba oil
Frankincense essential oil

### Beard Oil

Fractionated microbial olein (derivedfrom an oleaginous microorganism)
Jojoba oil
Cedarwood essential oil

### Facial Cleanser

Water
Myristic acid (derivedfrom an oleaginous microorganism)
Glycerin (derived from an oleaginous microorganism)
Behenic acid
Sodium methyl cocoyl taurate
Palmitic acid (derivedfrom an oleaginous microorganism)
Potassium hydroxide
Lauric acid (derivedfrom an oleaginous microorganism)
Stearic acid (derivedfrom an oleaginous microorganism)
Essential oils
PEG-3 distearate (derivedfrom an oleaginous microorganism)
Fragrance

### Shampoo

Water
Sodium laureth sulfate
Sodium citrate
Cocamidopropyl betaine
Cetearyl alcohol (derived from an oleaginous microorganism)
Sodium lauryl sulfate
Dimethiconol
Sodium chloride
Glycerin (derived from an oleaginous microorganism)
Fragrance
Avocado oil

### Conditioner

Water
Cetearyl alcohol (derived from an oleaginous microorganism)
Polysorbate 60 (derivedfrom an oleaginous microorganism)
Stearalkonium chloride
Jojoba seed oil
Microbial olein triglyceride oil (derivedfrom an oleaginous microorganism)
Potassium sorbate
Bran oil substitute (derivedfrom an oleaginous microorganism)
Sodium hyaluronate

### Baby Wash and Shampoo

Water
Sodium laureth sulfate
Cocamidopropyl hydroxsultaine
Cocamidopropyl betaine
Dimethiconol
Sodium chloride
Citric acid
Sodium benzoate
Glycerin (derived from an oleaginous microorganism)
Fragrance

### Hair Color

Water
p-Phenylenediamine
4-Chlororesorcinol
p-Aminophenol
Resorcinol
4-Amino-2-Hydroxytoluene
Sodium Hydrosulfite
Sodium Sulfite
Propylene glycol
Decyl Oleate (derivedfrom an oleaginous microorganism)
Beheneth-25 Methacrylate Copolymer
Ammonium Hydroxide

### Antiperspirant Deodorant

Aluminum chlorohydrate (active ingredient)
Cyclopentasiloxane
Isopropyl palmitate (derivedfrom an oleaginous microorganism)
Stearyl alcohol (derived from an oleaginous microorganism)
Whole microbial oil (derived from an oleaginous microorganism)
Talc
Hydrogenated castor oil
Fragrance

### Makeup

Mica
Octyldodecyl stearoyl stearate (derived from an oleaginous microorganism)
Dimethicone
Silica
Zinc stearate (derivedfrom an oleaginous microorganism)
Magnesium myristate (derivedfrom an oleaginous microorganism)
Rice starch
Methicone
Polymethyl methacrylate
Methylparaben
Sodium dehydroacetate
Alumina
Silk powder
Nylon-12
Propylparaben
Retinyl palmitate (derived from an oleaginous microorganism)
Titanium dioxide

### CONCLUSION

Based on the above analyses, the crude microbial oil described herein is a good match of palm oil/hybrid palm oil along a number of different parameters, demonstrating its suitability for use as an environmentally friendly alternative to plant-derived palm oil for use in personal care compositions.

### NUMBERED EMBODIMENTS OF THE INVENTION

Notwithstanding the appended claims, the disclosure sets forth the following numbered embodiments:
1. A refined, bleached, and/or deodorized (RBD) microbial oil produced by an oleaginous yeast.
2. A refined, bleached, and/or deodorized (RBD) microbial oil produced by an oleaginous yeast, wherein the microbial oil comprises ergosterol and does not comprise campesterol, β-sitosterol, or stigmasterol.
3. The microbial oil of embodiment 1 or 2, wherein a measurable amount of the pigment remains.
4. The microbial oil of embodiment 3, wherein the oil comprises at least one pigment selected from the group consisting of carotene, torulene and torulorhodin and does not comprise chlorophyll.
5. The microbial oil of any one of embodiments 1-4, wherein the oil is fractionable into two fractions, wherein the two fractions are microbial olein and microbial stearin, wherein each fraction comprises at least 10% of the microbial oil's original mass, and wherein the iodine value (IV) of the fractions differs by at least 10.
6. The microbial oil of any one of embodiments 1-5, wherein the oil has one or more characteristics similar to plant-derived palm oil selected from the group consisting of: apparent density, refractive index, saponification value, unsaponifiable matter, iodine value, slip melting point, and fatty acid composition.
7. A microbial oil produced by an oleaginous yeast, wherein the oil comprises a fatty acid profile comprising:
at least 30% w/w saturated fatty acids;
at least 30% w/w unsaturated fatty acids; and
less than 30% w/w total polyunsaturated fatty acids.
8. The microbial oil of embodiment 7, wherein the fatty acid profile comprises:
   greater than 40% w/w saturated fatty acids;
   greater than 40% w/w mono-unsaturated fatty acids; and
   less than 20% w/w polyunsaturated fatty acids.
9. The microbial oil of embodiment 7 or 8, wherein the saturated fatty acids have chain lengths between 16 and 18 carbons long.
10. The microbial oil of embodiment 7 or 8, wherein the unsaturated fatty acids have a chain length of 18 carbons.
11. The microbial oil of any one of embodiments 7-10, wherein the oil comprises ergosterol, at least 50 ppm ergosterol, or at least 100 ppm ergosterol.
12. The microbial oil of any one of embodiments 7-11, wherein the oil does not contain a phytosterol or chlorophyll.
13. The microbial oil of any one of embodiments 7-12, wherein the oil has one or more characteristics similar to plant-derived palm oil selected from the group consisting of iodine value, slip melting point, and overall saturation level.
14. The microbial oil of any one of embodiments 7-13, wherein the oil comprises 10-45% C16 saturated fatty acid.
15. The microbial oil of any one of embodiments 7-14, wherein the oil comprises 10-70% C18 unsaturated fatty acid.
16. The microbial oil of any one of embodiments 7-14, wherein the oil comprises 3-30% C18 saturated fatty acid.
17. The microbial oil of any one of embodiments 7-16, wherein the oil comprises a saponification value similar to that of plant-derived palm oil.
18. The microbial oil of any one of embodiments 7-17, wherein the oil comprises a saponification value of 150-210.
19. The microbial oil of any one of embodiments 7-18, wherein the oil comprises an iodine value similar to that of plant-derived palm oil.
20. The microbial oil of any one of embodiments 7-19, wherein the oil comprises an iodine value of 50-65.
21. The microbial oil of any one of embodiments 7-20, wherein the oil comprises a slip melting point similar to that of plant-derived palm oil.
22. The microbial oil of any one of embodiments 7-21, wherein the oil comprises a slip melting point of 20° C.-40° C.
23. The microbial oil of any one of embodiments 7-22, wherein the oil comprises a saturated fatty acid composition similar to that of plant-derived palm oil.
24. The microbial oil of any one of embodiments 7-23, wherein the oil comprises a saturated fatty acid composition of at most 70%.
25. The microbial oil of any one of embodiments 7-24, wherein the oil comprises an unsaturated fatty acid composition similar to that of plant-derived palm oil.
26. The microbial oil of any one of embodiments 7-25, wherein the oil comprises an unsaturated fatty acid composition of at most 70%.
27. The microbial oil of any one of embodiments 7-26, wherein the oil comprises less than 100 ppm of, comprises less than 50 ppm of, or does not comprise a phytosterol.
28. The microbial oil of embodiment 27, wherein the phytosterol is selected from the group consisting of campesterol, f3-sitosterol, stigmasterol.
29. The microbial oil of any one of embodiments 7-28, wherein the oil comprises less than 100 ppm of, comprises less than 50 ppm of, or does not comprise cholesterol.
30. The microbial oil of any one of embodiments 7-29, wherein the oil comprises less than 100 ppm of, comprises less than 50 ppm of, or does not comprise protothecasterol.
31. The microbial oil of any one of embodiments 7-30, wherein the oil does not comprise a pigment.
32. The microbial oil of any one of embodiments 7-30, wherein the oil comprises at least one pigment selected from the group consisting of β-carotene, torulene and torulorhodin.
33. The microbial oil of embodiment 32, wherein the oil comprises each of β-carotene, torulene and torulorhodin.
34. The microbial oil of any one of embodiments 7-33, wherein the oleaginous yeast is a recombinant yeast.
35. The microbial oil of any one of embodiments 7-34, wherein the oleaginous yeast is of the genus Yarrowia, Candida, Rhodotorula, Rhodosporidium, Metschnikowia, Cryptococcus, Trichosporon, or Lipomyces.
36. The microbial oil of any one of embodiments 7-35, wherein the oleaginous yeast is of the genus Rhodosporidium.
37. The microbial oil of any one of embodiments 7-36, wherein the oleaginous yeast is of the species Rhodosporidium toruloides.
38. The microbial oil of any one of embodiments 7-37, wherein the oil is fractionable.
39. The microbial oil of any one of embodiments 7-38, wherein the oil may be fractionated into microbial olein and microbial stearin.
40. The microbial oil of any one of embodiments 7-39, wherein the oil may be fractionated into microbial olein and microbial stearin, and wherein each fraction comprises at least 10% of the composition's starting mass.
41. The microbial oil of any one of embodiments 7-40, wherein the oil may be fractionated into microbial olein and microbial stearin, and wherein the iodine value (IV) of the fractions differs by at least 10.
42. The microbial oil of any one of embodiments 7-40, wherein the oil may be fractionated into microbial olein and microbial stearin, and wherein the IV of the fractions differs by at least 20.
43. The microbial oil of any one of embodiments 7-40, wherein the oil may be fractionated into microbial olein and microbial stearin, and wherein the IV of the fractions differs by at least 30.
44. The microbial oil of any one of embodiments 7-43, wherein the oil comprises at least 10% w/w C16 saturated fatty acid.
45. The microbial oil of any one of embodiments 7-44, wherein the oil comprises at least 10% w/w C18 unsaturated fatty acid.
46. The microbial oil of any one of embodiments 7-44, wherein the oil comprises at least 20% w/w C18 unsaturated fatty acid.
47. The microbial oil of any one of embodiments 7-46, wherein the oil comprises less than 5% combined C8, C10, C12, C14, and C15 saturated fatty acids.
48. The microbial oil of any one of embodiments 7-47, wherein the oil comprises:
   less than 10% w/w palmitic-palmitic-palmitic triglycerides;
   greater than 15% w/w palmitic-palmitic-oleic triglycerides; and
   greater than 15% w/w oleic-oleic-palmitic triglycerides.
49. The microbial oil of embodiment 48, wherein said palmitic-palmitic-palmitic triglyceride content is between about 0.8% and 1.3% w/w.
50. The microbial oil of embodiment 48, wherein said palmitic-palmitic-oleic triglyceride content is between about 16.9% and 28.2% w/w.
51. The microbial oil of embodiment 48, wherein said oleic-oleic-palmitic triglyceride content is between about 15.7% and 26.0% w/w.
52. The microbial oil of any one of embodiments 48-51, further comprising a stearic-stearic-oleic triglyceride content of less than 10% w/w and a stearic-oleic-oleic triglyceride content of less than 10% w/w.
53. The microbial oil of embodiment 52, wherein said stearic-stearic-oleic triglyceride content is between about 1.2% and 1.9% w/w.
54. The microbial oil of embodiment 52, wherein said stearic-oleic-oleic triglyceride content is between about 3.2% and 5.4% w/w.
55. The microbial oil of any one of embodiments 48-54, wherein greater than 40% of said triglycerides have one unsaturated sidechain, and wherein greater than 30% of said triglycerides have two unsaturated sidechains.
56. The microbial oil of any one of embodiments 48-55, wherein between 10% and 15% of palmitic and/or stearic fatty acids are located at the sn-2 position of triglyceride molecules.
57. The microbial oil of any one of embodiments 7-56, wherein the oil comprises the following amounts of fatty acids relative to the total fatty acids:
   between about 7.0% and 35% stearic acid;
   between about 10% and 50% oleic acid; and
   between about 8% and 20% linoleic acid.
58. The microbial oil of any one of embodiments 7-57, wherein the oil has been refined.
59. The microbial oil of embodiment 58, wherein the oil has been chemically and/or physically refined.
60. The microbial oil of embodiment 58 or 59, wherein the oil is deodorized.
61. The microbial oil of any one of embodiments 58-60, wherein the oil is bleached.
62. A derivative of the oil of any one of embodiments 1-61, wherein the derivative is a fraction, and wherein the fraction is microbial stearin, microbial olein, microbial soft mid-fraction, microbial super olein, microbial hard mid-fraction, microbial olein, and/or microbial top olein.
63. A personal care composition comprising:
   the microbial oil of any one of embodiments 1-62, and/or a derivative thereof, wherein the microbial oil is derived from an oleaginous yeast.
64. The composition of embodiment 63, wherein the microbial oil comprises a fatty acid profile of at least 30% saturation level.
65. The composition of embodiment 63 or 64, wherein the microbial oil comprises a fatty acid profile of at least 20% palmitic acid, at least 10% stearic acid, and at least 30% oleic acid.
66. The composition of any one of embodiments 63-65, wherein the microbial oil derivative is a triglyceride, diglyceride, monoglyceride, free fatty acid, fatty acid salt, glycerin, ester, fatty alcohol, fatty amine, derivatives thereof, or combination thereof.
67. The composition of any one of embodiments 63-66, wherein the microbial oil comprises at least one of ergosterol, β-carotene, torulene, and torularhodin.
68. The composition of any one of embodiments 63-67, wherein at least one of the oil derivatives functions as a surfactant in said personal care composition.
69. The composition of embodiment 68, wherein the surfactant is an emulsifier, detergent, wetting agent, foaming agent, thickening agent, emollient, pearlescent, solubilizer, conditioning agent, co-surfactant or dispersant in said personal care composition.
70. The composition of any one of embodiments 63-67, wherein at least one of the oil derivatives functions as a humectant in said personal care composition.
71. The composition of any one of embodiments 63-67, wherein the microbial oil or derivative thereof functions as a luxury soft oil in said personal care composition.
72. The composition of any one of embodiments 63-67, wherein the microbial oil or derivative thereof functions as a biologically active ingredient in said personal care composition.
73. The composition of any one of embodiments 63-72, further comprising a cleaning agent, a luxury soft oil, a polymer, an essential oil, a stabilizer, an emulsifier, a thickener, an antioxidant, a biologically active ingredient, or combinations thereof.
74. The composition of embodiment 73, wherein the emulsifier is a polysorbate, sorbitan ester, or polyethylene glycol.
75. The composition of embodiment 73, wherein the cleaning agent is an alkaline solution, acidic solution, neutral solution, degreaser, scouring agent, or combinations thereof.
76. The composition of embodiment 73, wherein the luxury soft oil is argan oil, jojoba oil, meadowfoam seed oil, seed oil, black seed oil, evening primrose oil, walnut oil, wheat germ oil, hemp oil, rosehip oil, pumpkin seed oil, or combinations thereof.
77. The composition of embodiment 73, wherein the essential oil is lavender, peppermint, tea tree oil, patchouli, eucalyptus, rhododendron, or combinations thereof.
78. The composition of embodiment 73, wherein the biologically active ingredient is an antibiotic, antimicrobial, anti-inflammatory, antioxidant, mineral, or inorganic compound derived from a mineral.
79. The composition of embodiment 73, wherein the biologically active ingredient is zinc oxide, retinol, or salicylic acid.
80. The composition of any one of embodiments 63-79, wherein the oleaginous yeast is Rhodosporidium toruloides.
81. The composition of any one of embodiments 63-80, wherein the composition does not comprise palm oil or palm kernel oil or derivatives thereof.
82. The composition of any one of embodiments 63-81, wherein the composition is a solid, liquid, cream, lotion, spray, gel, or foam.
83. The composition of any one of embodiments 63-82, wherein the composition is a skin repair serum.
84. The composition of any one of embodiments 63-82, wherein the composition is a cleansing oil.
85. The composition of any one of embodiments 63-82, wherein the composition is a lip balm.
86. The composition of any one of embodiments 63-82, wherein the composition is a lotion, balm, or cream.
87. The composition of any one of embodiments 63-82, wherein the composition is a makeup.
88. The composition of any one of embodiments 63-82, wherein the composition is a deodorant.
89. The composition of any one of embodiments 63-82, wherein the composition is a hair color.
90. The composition of any one of embodiments 63-82, wherein the composition is a baby wash and/or shampoo.
91. The composition of any one of embodiments 63-82, wherein the composition is a shampoo.
92. The composition of any one of embodiments 63-82, wherein the composition is a conditioner.
93. The composition of any one of embodiments 63-82, wherein the composition is a liquid cleanser or bar soap.
94. The composition of any one of embodiments 63-82, wherein the composition is a hair oil.
95. A personal care composition comprising an oil and/or derivative thereof, wherein said oil and/or derivative thereof consists of a microbial oil and/or derivative produced by an oleaginous yeast.
96. The composition of embodiment 95, wherein the microbial oil comprises a fatty acid profile of at least 30% saturation level.
97. The composition of embodiment 95 or 96, wherein the microbial oil comprises a fatty acid profile of at least 20% palmitic acid, at least 10% stearic acid, and at least 30% oleic acid.
98. The composition of any one of embodiments 95-97, wherein the composition comprises triglycerides, and wherein greater than 40% of said triglycerides have one unsaturated sidechain, and wherein greater than 30% of said triglycerides have two unsaturated sidechains.
99. The composition of any one of embodiments 95-98, wherein the microbial oil comprises at least one of ergosterol, β-carotene, torulene, and torularhodin.
100. The composition of any one of embodiments 95-99, wherein the microbial oil derivative is a triglyceride, diglyceride, monoglyceride, free fatty acid, fatty acid salt, glycerin, ester, fatty alcohol, fatty amine, derivatives thereof, or combination thereof.
101. The composition of any one of embodiments 95-100, wherein at least one of the oil derivatives functions as a surfactant, biologically active ingredient, or humectant in said personal care composition.
102. The composition of embodiment 101, wherein the surfactant is an emulsifier, detergent, wetting agent, foaming agent, thickening agent, emollient, pearlescent, solubilizer, conditioning agent, co-surfactant or dispersant in said personal care composition.
103. The composition of any one of embodiments 95-102, wherein the microbial oil or derivative thereof functions as a luxury soft oil in said personal care composition.
104. The composition of any one of embodiments 95-103, wherein the oleaginous yeast is Rhodosporidium toruloides.
105. The composition of any one of embodiments 95-104, wherein the composition is a solid, liquid, cream, lotion, spray, gel, or foam.
106. A method for producing a personal care composition, comprising:
   obtaining a microbial oil, and/or a derivative thereof, wherein the microbial oil and/or derivative thereof is derived from an oleaginous yeast; and
   producing a personal care composition.
107. The method of embodiment 106 wherein the microbial oil comprises a fatty acid profile of at least 30% saturation level.
108. The method of embodiment 106 or 107, wherein the microbial oil comprises a fatty acid profile of at least 20% palmitic acid, at least 10% stearic acid, and at least 30% oleic acid.
109. The method of any one of embodiments 106-108, wherein the microbial oil is refined, bleached, and/or deodorized.
110. The method of any one of embodiments 106-109, wherein the microbial oil derivative is a triglyceride, diglyceride, monoglyceride, free fatty acid, fatty acid salt, glycerin, ester, fatty alcohol, fatty amine, derivatives thereof, or combination thereof.
111. The method of any one of embodiments 106-110, wherein the microbial oil comprises at least one of ergosterol, β-carotene, torulene, and torularhodin.
112. The method of any one of embodiments 106-111, wherein at least one of the oil derivatives functions as a surfactant in said personal care composition.
113. The method of embodiment 112, wherein the surfactant is an emulsifier, detergent, wetting agent, foaming agent, thickening agent, emollient, pearlescent, solubilizer, conditioning agent, co-surfactant or dispersant in said personal care composition.
114. The method of any one of embodiments 106-111, wherein at least one of the oil derivatives functions as a humectant in said personal care composition.
115. The method of any one of embodiments 106-114, wherein the microbial oil or derivative thereof functions as a luxury soft oil in said personal care composition.
116. The method of any one of embodiments 106-115, wherein the personal care composition further comprises a cleaning agent, a luxury soft oil, a polymer, an essential oil, a stabilizer, an emulsifier, a thickener, an antioxidant, a biologically active ingredient, or combinations thereof.
117. The method of any one of embodiments 106-116, wherein the oleaginous yeast is Rhodosporidium toruloides.
118. The method of any one of embodiments 106-117, wherein the composition does not comprise palm oil or palm kernel oil or derivatives thereof.
119. A personal care composition produced by the method of any one of embodiments 106-118, wherein the composition is a solid, liquid, cream, lotion, spray, gel, or foam.
120. A personal care composition produced by any one of embodiments 106-119, wherein the personal care item is a soap, body lotion, face lotion, luxury soft oil, cleansing oil, cream, deodorant, or hair care item.
121. A personal care composition comprising sodium stearate derived from a stearic acid produced by an oleaginous yeast.
122. A personal care composition comprising a fatty acid-ingredient derived from an oleaginous yeast.
123. A personal care composition comprising cetearyl alcohol derived from fatty alcohols produced by an oleaginous yeast.
124. A personal care composition comprising an isostearyl palmitate derived from an oleaginous yeast.
125. A method for producing a derivative of a microbial oil comprising:
   obtaining a whole cell or lysed microbial biomass;
   extracting crude microbial oil from the whole cell or lysed microbial biomass, wherein said extraction process removes toxins and produces a microbial oil safe for human use; and
   modifying the microbial oil, wherein said modifying comprises fractionation, interesterification, transesterification, hydrogenation, steam hydrolysis, distillation, saponification, or combinations thereof.
126. The method of embodiment 125, wherein the method further comprises at least one of physically refining, chemically refining, deodorizing and bleaching the microbial oil.
127. The method of embodiment 125 or 126, wherein the method produces a derivative selected from a triglyceride, diglyceride, monoglyceride, free fatty acid, fatty acid salt, glycerin, ester, fatty alcohol, fatty amine, derivative thereof, and combinations thereof.
128. The method of any one of embodiments 125-127, wherein the whole cell or lysed microbial biomass is produced by Rhodosporidium toruloides.
129. The method of embodiment 125, wherein the crude microbial oil extracted from the whole cell or lysed microbial biomass is as described in any one of embodiments 7-59.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgement or any form of suggestion that they constitute valid prior art or form part of the common general knowledge in any country in the world. The following international PCT publications are incorporated herein by reference in their entirety: International Patent Publication No. WO2021/154863 and WO2021/163194.

What is claimed is:

1. A refined, bleached, and/or deodorized (RBD) microbial oil produced by an oleaginous yeast.

2. A refined, bleached, and/or deodorized (RBD) microbial oil produced by an oleaginous yeast, wherein the microbial oil comprises ergosterol and does not comprise campesterol, β-sitosterol, or stigmasterol.

3. The microbial oil of claim 1 or 2, wherein a measurable amount of the pigment remains.

4. The microbial oil of claim 3, wherein the oil comprises at least one pigment selected from the group consisting of carotene, torulene and torulorhodin and does not comprise chlorophyll.

5. The microbial oil of claim 1, wherein the oil is fractionable into two fractions, wherein the two fractions are microbial olein and microbial stearin, wherein each fraction comprises at least 10% of the microbial oil's original mass, and wherein the iodine value (IV) of the fractions differs by at least 10.

6. The microbial oil of claim 1, wherein the oil has one or more characteristics similar to plant-derived palm oil selected from the group consisting of: apparent density, refractive index, saponification value, unsaponifiable matter, iodine value, slip melting point, and fatty acid composition.

7. A microbial oil produced by an oleaginous yeast, wherein the oil comprises a fatty acid profile comprising:
at least 30% w/w saturated fatty acids;
at least 30% w/w unsaturated fatty acids; and
less than 30% w/w total polyunsaturated fatty acids.

8. The microbial oil of claim 7, wherein the fatty acid profile comprises:
greater than 40% w/w saturated fatty acids;
greater than 40% w/w mono-unsaturated fatty acids; and
less than 20% w/w polyunsaturated fatty acids.

9. The microbial oil of claim 7, wherein the saturated fatty acids have chain lengths between 16 and 18 carbons long.

10. The microbial oil of claim 7, wherein the unsaturated fatty acids have a chain length of 18 carbons.

11. The microbial oil of claim 7, wherein the oil comprises ergosterol, at least 50 ppm ergosterol, or at least 100 ppm ergosterol.

12. The microbial oil of claim 7, wherein the oil does not contain a phytosterol or chlorophyll.

13. The microbial oil of claim 7, wherein the oil has one or more characteristics similar to plant-derived palm oil selected from the group consisting of iodine value, slip melting point, and overall saturation level.

14. The microbial oil of claim 7, wherein the oil comprises 10-45% C16 saturated fatty acid.

15. The microbial oil of claim 7, wherein the oil comprises 10-70% C18 unsaturated fatty acid.

16. The microbial oil of claim 7, wherein the oil comprises 3-30% C18 saturated fatty acid.

17. The microbial oil of claim 7, wherein the oil comprises a saponification value similar to that of plant-derived palm oil.

18. The microbial oil of claim 7, wherein the oil comprises a saponification value of 150-210.

19. The microbial oil of claim 7, wherein the oil comprises an iodine value similar to that of plant-derived palm oil.

20. The microbial oil of claim 7, wherein the oil comprises an iodine value of 50-65.

21. The microbial oil of claim 7, wherein the oil comprises a slip melting point similar to that of plant-derived palm oil.

22. The microbial oil of claim 7, wherein the oil comprises a slip melting point of 20° C.-40° C.

23. The microbial oil of claim 7, wherein the oil comprises a saturated fatty acid composition similar to that of plant-derived palm oil.

24. The microbial oil of claim 7, wherein the oil comprises a saturated fatty acid composition of at most 70%.

25. The microbial oil of claim 7, wherein the oil comprises an unsaturated fatty acid composition similar to that of plant-derived palm oil.

26. The microbial oil of claim 7, wherein the oil comprises an unsaturated fatty acid composition of at most 70%.

27. The microbial oil of claim 7, wherein the oil comprises less than 100 ppm of, comprises less than 50 ppm of, or does not comprise a phytosterol.

28. The microbial oil of claim 27, wherein the phytosterol is selected from the group consisting of campesterol, β-sitosterol, stigmasterol.

29. The microbial oil of claim 7, wherein the oil comprises less than 100 ppm of, comprises less than 50 ppm of, or does not comprise cholesterol.

30. The microbial oil of claim 7, wherein the oil comprises less than 100 ppm of, comprises less than 50 ppm of, or does not comprise protothecasterol.

31. The microbial oil of claim 7, wherein the oil does not comprise a pigment.

32. The microbial oil of claim 7, wherein the oil comprises at least one pigment selected from the group consisting of β-carotene, torulene and torulorhodin.

33. The microbial oil of claim 32, wherein the oil comprises each of β-carotene, torulene and torulorhodin.

34. The microbial oil of claim 7, wherein the oleaginous yeast is a recombinant yeast.

35. The microbial oil of claim 7, wherein the oleaginous yeast is of the genus Yarrowia, Candida, Rhodotorula, Rhodosporidium, Metschnikowia, Cryptococcus, Trichosporon, or Lipomyces.

36. The microbial oil of claim 7, wherein the oleaginous yeast is of the genus Rhodosporidium.

37. The microbial oil of claim 7, wherein the oleaginous yeast is of the species Rhodosporidium toruloides.

38. The microbial oil of claim 7, wherein the oil is fractionable.

39. The microbial oil of claim 7, wherein the oil may be fractionated into microbial olein and microbial stearin.

40. The microbial oil of claim 7, wherein the oil may be fractionated into microbial olein and microbial stearin, and wherein each fraction comprises at least 10% of the composition's starting mass.

41. The microbial oil of claim 7, wherein the oil may be fractionated into microbial olein and microbial stearin, and wherein the iodine value (IV) of the fractions differs by at least 10.

42. The microbial oil of claim 7, wherein the oil may be fractionated into microbial olein and microbial stearin, and wherein the IV of the fractions differs by at least 20.

43. The microbial oil of claim 7, wherein the oil may be fractionated into microbial olein and microbial stearin, and wherein the IV of the fractions differs by at least 30.

44. The microbial oil of claim 7, wherein the oil comprises at least 10% w/w C16 saturated fatty acid.

45. The microbial oil of claim 7, wherein the oil comprises at least 10% w/w C18 unsaturated fatty acid.

46. The microbial oil of claim 7, wherein the oil comprises at least 20% w/w C18 unsaturated fatty acid.

47. The microbial oil of claim 7, wherein the oil comprises less than 5% combined C8, C10, C12, C14, and C15 saturated fatty acids.

48. The microbial oil of claim 7, wherein the oil comprises:
less than 10% w/w palmitic-palmitic-palmitic triglycerides;
greater than 15% w/w palmitic-palmitic-oleic triglycerides; and
greater than 15% w/w oleic-oleic-palmitic triglycerides.

49. The microbial oil of claim 48, wherein said palmitic-palmitic-palmitic triglyceride content is between about 0.8% and 1.3% w/w.

50. The microbial oil of claim 48, wherein said palmitic-palmitic-oleic triglyceride content is between about 16.9% and 28.2% w/w.

51. The microbial oil of claim 48, wherein said oleic-oleic-palmitic triglyceride content is between about 15.7% and 26.0% w/w.

52. The microbial oil of claim 48, further comprising a stearic-stearic-oleic triglyceride content of less than 10% w/w and a stearic-oleic-oleic triglyceride content of less than 10% w/w.

53. The microbial oil of claim 52, wherein said stearic-stearic-oleic triglyceride content is between about 1.2% and 1.9% w/w.

54. The microbial oil of claim 52, wherein said stearic-oleic-oleic triglyceride content is between about 3.2% and 5.4% w/w.

55. The microbial oil of claim 48, wherein greater than 40% of said triglycerides have one unsaturated sidechain, and wherein greater than 30% of said triglycerides have two unsaturated sidechains.

56. The microbial oil of claim 48, wherein between 10% and 15% of palmitic and/or stearic fatty acids are located at the sn-2 position of triglyceride molecules.

57. The microbial oil of claim 7, wherein the oil comprises the following amounts of fatty acids relative to the total fatty acids:
between about 7.0% and 35% stearic acid;
between about 10% and 50% oleic acid; and
between about 8% and 20% linoleic acid.

58. The microbial oil of claim 7, wherein the oil has been refined.

59. The microbial oil of claim 58, wherein the oil has been chemically and/or physically refined.

60. The microbial oil of claim 58, wherein the oil is deodorized.

61. The microbial oil of claim 58, wherein the oil is bleached.

62. A derivative of the oil of claim 7, wherein the derivative is a fraction, and wherein the fraction is microbial stearin, microbial olein, microbial soft mid-fraction, microbial super olein, microbial hard mid-fraction, microbial olein, and/or microbial top olein.

63. A personal care composition comprising:
the microbial oil of claim 7, and/or a derivative thereof, wherein the microbial oil is derived from an oleaginous yeast.

64. The composition of claim 63, wherein the microbial oil comprises a fatty acid profile of at least 30% saturation level.

65. The composition of claim 63, wherein the microbial oil comprises a fatty acid profile of at least 20% palmitic acid, at least 10% stearic acid, and at least 30% oleic acid.

66. The composition of claim 63, wherein the microbial oil derivative is a triglyceride, diglyceride, monoglyceride, free fatty acid, fatty acid salt, glycerin, ester, fatty alcohol, fatty amine, derivatives thereof, or combination thereof.

67. The composition of claim 63, wherein the microbial oil comprises at least one of ergosterol, β-carotene, torulene, and torularhodin.

68. The composition of claim 63, wherein at least one of the oil derivatives functions as a surfactant in said personal care composition.

69. The composition of claim 68, wherein the surfactant is an emulsifier, detergent, wetting agent, foaming agent, thickening agent, emollient, pearlescent, solubilizer, conditioning agent, co-surfactant or dispersant in said personal care composition.

70. The composition of claim 63, wherein at least one of the oil derivatives functions as a humectant in said personal care composition.

71. The composition of claim 63, wherein the microbial oil or derivative thereof functions as a luxury soft oil in said personal care composition.

72. The composition of claim 63, wherein the microbial oil or derivative thereof functions as a biologically active ingredient in said personal care composition.

73. The composition of claim 63, further comprising a cleaning agent, a luxury soft oil, a polymer, an essential oil, a stabilizer, an emulsifier, a thickener, an antioxidant, a biologically active ingredient, or combinations thereof.

74. The composition of claim 73, wherein the emulsifier is a polysorbate, sorbitan ester, or polyethylene glycol.

75. The composition of claim 73, wherein the cleaning agent is an alkaline solution, acidic solution, neutral solution, degreaser, scouring agent, or combinations thereof.

76. The composition of claim 73, wherein the luxury soft oil is argan oil, jojoba oil, meadowfoam seed oil, seed oil, black seed oil, evening primrose oil, walnut oil, wheat germ oil, hemp oil, rosehip oil, pumpkin seed oil, or combinations thereof.

77. The composition of claim 73, wherein the essential oil is lavender, peppermint, tea tree oil, patchouli, eucalyptus, rhododendron, or combinations thereof.

78. The composition of claim 73, wherein the biologically active ingredient is an antibiotic, antimicrobial, anti-inflammatory, antioxidant, mineral, or inorganic compound derived from a mineral.

79. The composition of claim 73, wherein the biologically active ingredient is zinc oxide, retinol, or salicylic acid.

80. The composition of claim 63, wherein the oleaginous yeast is Rhodosporidium toruloides.

81. The composition of claim 63, wherein the composition does not comprise palm oil or palm kernel oil or derivatives thereof.

82. The composition of claim 63, wherein the composition is a solid, liquid, cream, lotion, spray, gel, or foam.

83. The composition of claim 63, wherein the composition is a skin repair serum.

84. The composition of claim 63, wherein the composition is a cleansing oil.

85. The composition of claim 63, wherein the composition is a lip balm.

86. The composition of claim 63, wherein the composition is a lotion, balm, or cream.

87. The composition of claim 63, wherein the composition is a makeup.

88. The composition of claim 63, wherein the composition is a deodorant.

89. The composition of claim 63, wherein the composition is a hair color.

90. The composition of claim 63, wherein the composition is a baby wash and/or shampoo.

91. The composition of claim 63, wherein the composition is a shampoo.

92. The composition of claim 63, wherein the composition is a conditioner.

93. The composition of claim 63, wherein the composition is a liquid cleanser or bar soap.

94. The composition of claim 63, wherein the composition is a hair oil.

95. A personal care composition comprising an oil and/or derivative thereof, wherein said oil and/or derivative thereof consists of a microbial oil and/or derivative produced by an oleaginous yeast.

96. The composition of claim 95, wherein the microbial oil comprises a fatty acid profile of at least 30% saturation level.

97. The composition of claim 95, wherein the microbial oil comprises a fatty acid profile of at least 20% palmitic acid, at least 10% stearic acid, and at least 30% oleic acid.

98. The composition of claim 95, wherein the composition comprises triglycerides, and wherein greater than 40% of said triglycerides have one unsaturated sidechain, and wherein greater than 30% of said triglycerides have two unsaturated sidechains.

99. The composition of claim 95, wherein the microbial oil comprises at least one of ergosterol, β-carotene, torulene, and torularhodin.

100. The composition of claim 95, wherein the microbial oil derivative is a triglyceride, diglyceride, monoglyceride, free fatty acid, fatty acid salt, glycerin, ester, fatty alcohol, fatty amine, derivatives thereof, or combination thereof.

101. The composition of claim 95, wherein at least one of the oil derivatives functions as a surfactant, biologically active ingredient, or humectant in said personal care composition.

102. The composition of claim 101, wherein the surfactant is an emulsifier, detergent, wetting agent, foaming agent, thickening agent, emollient, pearlescent, solubilizer, conditioning agent, co-surfactant or dispersant in said personal care composition.

103. The composition of claim 95, wherein the microbial oil or derivative thereof functions as a luxury soft oil in said personal care composition.

104. The composition of claim 95, wherein the oleaginous yeast is Rhodosporidium toruloides.

105. The composition of claim 95, wherein the composition is a solid, liquid, cream, lotion, spray, gel, or foam.

106. A method for producing a personal care composition, comprising:
obtaining a microbial oil, and/or a derivative thereof, wherein the microbial oil and/or derivative thereof is derived from an oleaginous yeast; and
producing a personal care composition.

107. The method of claim 106 wherein the microbial oil comprises a fatty acid profile of at least 30% saturation level.

108. The method of claim 106, wherein the microbial oil comprises a fatty acid profile of at least 20% palmitic acid, at least 10% stearic acid, and at least 30% oleic acid.

109. The method of claim 106, wherein the microbial oil is refined, bleached, and/or deodorized.

110. The method of claim 106, wherein the microbial oil derivative is a triglyceride, diglyceride, monoglyceride, free fatty acid, fatty acid salt, glycerin, ester, fatty alcohol, fatty amine, derivatives thereof, or combination thereof.

111. The method of claim 106, wherein the microbial oil comprises at least one of ergosterol, β-carotene, torulene, and torularhodin.

112. The method of claim 106, wherein at least one of the oil derivatives functions as a surfactant in said personal care composition.

113. The method of claim 112, wherein the surfactant is an emulsifier, detergent, wetting agent, foaming agent, thickening agent, emollient, pearlescent, solubilizer, conditioning agent, co-surfactant or dispersant in said personal care composition.

114. The method of claim 106, wherein at least one of the oil derivatives functions as a humectant in said personal care composition.

115. The method of claim 106, wherein the microbial oil or derivative thereof functions as a luxury soft oil in said personal care composition.

116. The method of claim 106, wherein the personal care composition further comprises a cleaning agent, a luxury soft oil, a polymer, an essential oil, a stabilizer, an emulsifier, a thickener, an antioxidant, a biologically active ingredient, or combinations thereof.

117. The method of claim 106, wherein the oleaginous yeast is Rhodosporidium toruloides.

118. The method of claim 106, wherein the composition does not comprise palm oil or palm kernel oil or derivatives thereof.

119. A personal care composition produced by the method of claim 106, wherein the composition is a solid, liquid, cream, lotion, spray, gel, or foam.

120. A personal care composition produced by claim 106, wherein the personal care item is a soap, body lotion, face lotion, luxury soft oil, cleansing oil, cream, deodorant, or hair care item.

121. A personal care composition comprising sodium stearate derived from a stearic acid produced by an oleaginous yeast.

122. A personal care composition comprising a fatty acid-ingredient derived from an oleaginous yeast.

123. A personal care composition comprising cetearyl alcohol derived from fatty alcohols produced by an oleaginous yeast.

124. A personal care composition comprising an isostearyl palmitate derived from an oleaginous yeast.

125. A method for producing a derivative of a microbial oil comprising:
obtaining a whole cell or lysed microbial biomass;
extracting crude microbial oil from the whole cell or lysed microbial biomass, wherein said extraction process removes toxins and produces a microbial oil safe for human use; and
modifying the microbial oil, wherein said modifying comprises fractionation, interesterification, transesterification, hydrogenation, steam hydrolysis, distillation, saponification, or combinations thereof.

126. The method of claim 125, wherein the method further comprises at least one of physically refining, chemically refining, deodorizing and bleaching the microbial oil.

127. The method of claim 125, wherein the method produces a derivative selected from a triglyceride, diglyceride, monoglyceride, free fatty acid, fatty acid salt, glycerin, ester, fatty alcohol, fatty amine, derivative thereof, and combinations thereof.

128. The method of claim 125, wherein the whole cell or lysed microbial biomass is produced by Rhodosporidium toruloides.

129. The method of claim 125, wherein the crude microbial oil extracted from the whole cell or lysed microbial biomass is as described in any one of claims 7-59.
